(19) United States
(12) Patent Application Publication
(10) Pub. No.: US 2009/0234396 A1
(43) Pub. Date: Sep. 17, 2009

(54) METHOD AND APPARATUS FOR ARTICULAR SCAPHOLUNATE RECONSTRUCTION

(75) Inventor: Robert J. Medoff, Kailua, HI (US)

Correspondence Address: PATZIK, FRANK & SAMOTNY LTD., 150 SOUTH WACKER DRIVE, SUITE 1500, CHICAGO, IL, 60606, US

(73) Assignees: Lars G. Tellman, Falsterbo (SE); David Medoff, Valencia, CA (US)

(21) Appl. No.: 12/394,090

(22) Filed: Feb. 27, 2009

Related U.S. Application Data
(60) Provisional application No. 61/032,515, filed on Feb. 29, 2008.

(51) Int. Cl. A61F 5/00; A61F 2/42; A61B 17/58

(52) U.S. Cl. 606/86 R; 623/21.12; 606/96

(57) ABSTRACT

A reconstructive procedure for addressing instability of the scapholunate articulation between a scaphoid and a lunate of the hand. A graft is obtained. At least a portion of the graft is positioned intramedullary to the scaphoid. At least another portion of the graft is positioning intramedullary to the lunate. The graft crosses directly through the scapholunate joint. A plurality of instruments and implements, including an E-shaped drill guide, a cannulated lunate screw, and suture anchors in the form of button-shaped or bead-shaped anchors, are provided to facilitate the surgeon's performance of the scapholunate ligament reconstruction. A kit is provided, including a plurality of instruments and implements for performing the reconstructive procedure.

### CROSS-REFERENCE TO RELATED APPLICATIONS

This application claims the benefit of U.S. provisional application No. 61/032,515, filed Feb. 29, 2008, the entirety of which is hereby incorporated by reference.

### BACKGROUND OF THE INVENTION

1. Field of the Invention

The invention relates, in general, to reconstruction following injury to the scapholunate joint of the human hand and, more particularly, to methods and surgical instruments for reconstructing the scapholunate joint.

2. Description of Related Art

The human wrist is a complex articulation that allows motion in multiple planes. The wrist is actually a composite of multiple joint surfaces present between the distal end of the radius and ulna and the eight intrinsic carpal bones. On the proximal side of the joint are the broad joint surfaces formed from the distal end of the radius and ulna. Within the center of the wrist are two rows of small carpal bones, a proximal carpal row formed by the lunate, triquetrum, and pisiform and a distal carpal row formed by the trapezium, trapezoid, capitate, and hamate. Linking the two rows on the radial side of the carpus is the eighth carpal bone, the scaphoid. All of these bone elements are connected by a complex organization of multiple ligaments that provide stability of the multiple joint surfaces yet allow controlled motion to occur between the various osseous elements.

The joint that is formed between the base of the scaphoid and the lunate is particularly important. The lunate is a crescent shaped bone that is one of the main areas of load transfer between the proximal carpal row of the wrist and the distal radius. The scaphoid is an oblong bone that acts as a linkage between the proximal carpal row and the distal carpal row. Proximally, the scaphoid has a relatively flat surface where it articulates with an adjacent flat surface on the radial side of the lunate. The base of the scaphoid and lunate are anchored to each other by a group of ligaments known collectively as the scapholunate interosseous ligaments. These ligaments form a C-shaped connection between the base of the scaphoid and lunate, extending from the dorsal surface of this joint, continuing as a flat connecting ligament along the curved proximal edge where the two bones come together and ending in a palmar connection volarly.

Under normal conditions, the ligaments that connect the base of the scaphoid to the lunate function to coordinate motion between these bones. This prevents gapping between the scaphoid and lunate, prevents rotation of the scaphoid along its long axis (pronation or supination in relation to the lunate), and allows only slight dorsal or palmar translation of the base of the scaphoid in relation to the lunate. The C-shaped scapholunate interosseous ligament that extends from the dorsal edge back along the curved proximal edge of the articular surface between the two bones and ending at the distal palmar edge is a functional constraint that prevents step-off of the articular surface across the two bones. In effect, one role of these ligaments is to maintain a smooth articular surface between the base of the scaphoid and the proximal surface of the lunate so that abnormal wear of the distal radius is prevented. However, the inventor has observed that normally these ligaments function to allow the scaphoid to flex in relation to the lunate along a nearly circular arc of motion of the base of the scaphoid in relation to the lunate. These requirements of maintaining a congruent proximal articular surface between the scaphoid and lunate, yet simultaneously allowing flexion/extension movements of the scaphoid in relation to the lunate, is necessary to normal function of the wrist joint.

The C-shaped structure of the scapholunate interosseous ligaments is important in allowing flexion/extension while preventing translation or rotation of the scaphoid along its long axis. The C-shaped form results in a nearly transverse axis of rotation that is essentially perpendicular to the plane of the scapholunate joint at the center of the base of the scaphoid in relation to the lunate so that the base of the scaphoid can only displace slightly dorsally or palmarly with flexion/extension of the wrist. In addition, these ligaments constrain translational movements. It is the inventor's belief that the entire scapholunate interosseous complex is necessary for normal motion of the scaphoid.

The prevalent current teaching, though one that the inventor does not necessarily agree with, is that the dorsal edge of the scapholunate interosseous ligament is the main stabilizer of the scapholunate joint, and reconstruction of this portion of the ligament is all that is needed to restore normal or nearly normal joint function. If the only ligamentous attachment between the scaphoid and lunate were at the dorsal margin, the axis of rotation of the scaphoid with flexion and extension would be changed and shifted dorsally, resulting in dorsal translation of the base of the scaphoid as it flexes in relation to the base of the lunate. This would be expected to result in abnormal kinematics or movement of the scaphoid that contribute to osteoarthritis of the joint. In fact, in cases of chronic scapholunate disruption, accelerated osteoarthritis of the wrist ensues, and is usually first apparent at the articulation between the scaphoid and the distal articular surface of the radius. In these cases, the scaphoid can be noted to be translated dorsally and in a hyperflexed position, resulting in an incongruent joint with force concentration at the dorsal rim of the radius. This abnormal position is a result of the abnormal dorsal translation of the base of the scaphoid that occurs with this ligament injury.

As might be expected from the preceding description, injuries that result in rupture of the scapholunate interosseous ligament often lead to marked disability and arthritis of the wrist. In this context, the connection between the base of the scaphoid and the lunate is disrupted, allowing asynchronous motion to occur between these two small carpal bones. Typically, this results in separation of the base of the scaphoid away from the lunate, and migration of the capitate between these two bones. In this situation, the base of the scaphoid is no longer constrained to maintain congruency with the proximal articular surface of the lunate, but can displace dorsally resulting in significant offset of the articular surface and causing it to ride up against the dorsal edge of the articular surface of the radius. In addition, the scaphoid becomes uncoupled rotationally, resulting in pronation of the base and further incongruity of the radiocarpal joint. These abnormalities result in altered kinematics of the wrist and lead to an inexorable progression of pain, limited motion, decreased function, and arthritis.

Typically, significant rupture of the scapholunate interosseous ligament can result in subtle but consistent abnormalities of the relative positions of the carpal bones that can be identified on regular X-rays. The finding of an extended lunate in combination with a flexed scaphoid on the lateral film are suggestive of a scapholunate interosseous ligament tear. In addition, the finding of a gap between the scaphoid and lunate, sometimes only noted on a clenched fist view to load the wrist, can occur with this injury. Since rupture of the scapholunate interosseous ligament results in loss of the scaphoid linkage between the proximal carpal row and the distal carpal row resulting in a dorsiflexed attitude of the lunate, this condition has been also described as a dorsal intercalated segmental instability pattern, or DISI.

Many attempts to restore stability to the scapholunate joint have been tried over the years. One approach has been attempts at simple repair. Unfortunately, these ligaments are extremely short, often only a millimeter or two in length, and are usually shredded beyond repair. Furthermore, these bones are small and mostly covered by articular cartilage; it is technically difficult to place sutures directly into the small non-articular regions of bone. Sutures into the remaining ligament are tenuous and often rip out of the tiny ligament remnant that remains. Because fixation is tenuous, it must be supplemented with immobilization of the carpal bones with multiple pins for several weeks. Typically, this approach results in residual instability of the scapholunate joint and loss of movement.

A different approach has been reconstruction of the scapholunate joint with fully extramedullary placement of a tendon or ligament from another source. Tendons from the flexor carpi radialis, palmaris longus, and even bone-ligament-bone preparations that are harvested from the carpal-metacarpal joints of the hand or tarsal-metatarsal joints of the foot have been tried. In addition, allograft preparations (from a cadaveric human donor) have been tried. In general, these techniques have only reconstructed the dorsal scapholunate ligament by positioning and fixing the graft to the dorsal non-articular surface of the scaphoid and the lunate (or sometimes triquetrum). However, the available bone in this area is extremely small, compromising fixation by providing only a very limited area of ligament attachment and making this procedure technically difficult. In addition, since only the dorsal ligament is reconstructed, the rotational axis of the scaphoid in relation to the lunate is altered and moved dorsally, resulting in abnormal motion that causes the base of the scaphoid to shift dorsally as it flexes. In addition, the scaphoid and lunate remain uncoupled from rotational movement in pronation/supination. Again, since fixation of the tendon grafts to the bone elements is tenuous, fixation must be supplemented with temporary pins across the joints and prolonged immobilization that can result in stiffness. These issues often result in residual dysfunction, progression of arthritis, and a suboptimal result.

More recently, an approach to DISI instability caused by scapholunate ligament tears has been advocated which is characterized by inserting a screw across the scaphoid into the lunate. This so called RASL (‘reduction anatomic of the scapho-lunate’) procedure uses the screw to reduce and constrain the gap between the two bones. The screw is a rigid connection between the scaphoid and lunate. However, since the scaphoid “wants to flex” in relation to the lunate during normal motion, this procedure destroys the normal kinematics between these two bones. Because the screw is rigid, normal movements load the screw to create ‘windshield wiper’ movements in the scaphoid from the torque on the screw; this can result in significant bone loss. If the screw is not placed precisely in the correct axis of rotation, motion is restricted. In addition, if the scaphoid is allowed to move in relation to the lunate, some motion between the screw and the bone must occur. This often leads to destruction of the bone by movement against the screw and can lead to migration into the joint, fracture, arthritis, or breakage of hardware. Although more recently, screws have been introduced that attempt to allow some degree of rotation between the proximal and distal section of the screw in order to avoid these problems, this requires small moving parts that are subject to breakage with hardware that is more difficult to insert. In addition, since most implants are susceptible to fatigue and eventual failure, it is not a long term solution, particular for the younger patient population in whom this injury is commonly seen.

The present invention is intended to overcome these problems, and seeks to restore nearly normal kinematics to the scapholunate joint. One object of the present invention is to create a ligament reconstruction that limits dorsal/palmar translation of the base of the scaphoid in relation to the lunate, prevents separation or gapping of the scapholunate articulation, limits rotational movement of the scaphoid along its long axis (into pronation/supination relative to the lunate), yet allows near normal flexion/extension of the scaphoid relative to the lunate and this arc of motion along a physiologic transverse axis of rotation.

Another object of the present invention is to provide a means for a stable ligament reconstruction that is strong enough to allow early rehabilitation without require pinning of the carpus or prolonged periods of immobilization. Another object of the present invention is to provide a means to achieve strong fixation of a ligament reconstruction in a bone that is extremely small and predominantly articular. Another object of the present invention is to get a very strong repair in these very small bones, one that hopefully does not require pinning of the joint or immobilization; this is very difficult to do with techniques that simply apply a graft to the dorsal edges of these bones.

Another object of the present invention is to create a reconstruction that restores stability to prevent dorsal subluxation of the base of the scaphoid, which may well be the predominant cause of arthritis in injuries to the scapholunate joint. Another object of the present invention is to prevent gapping between the scaphoid and lunate, and to prevent abnormal rotation of the scaphoid along its long axis (pronation/supination), while at the same time allowing the normal physiologic flexion/extension of the scaphoid in relation to the lunate. Another object of the present invention is to restore the normal axis of rotation of the scaphoid for flexion/extension in relation to the lunate. Another objective of the present invention is a ligament reconstruction that has the ability to develop into a living, vascularized ligament that is not prone to implant failure with long term use.

These and other objects and features of the present invention will become apparent in view of the present specification, drawings and claims.

### BRIEF SUMMARY OF THE INVENTION

The present invention includes methods for performing articular scapholunate ligament reconstruction using a tendon graft, as well as implements and instruments, including guides, screws, and suture anchors in the form of button-shaped and bead-shaped anchors, for facilitating a surgeon's performance of the various methods of the present invention. Moreover, the button-shaped and bead-shaped suture anchors of the present application are considered to have relatively broad surgical application, apart from scapholunate reconstruction. The present invention further includes kits comprising combinations of several of these implements and instruments.

In one method of the present invention, a tendon graft is harvested from the palmar longus or another suitable tendon. After exposing the carpus to the degree required, the scapholunate joint is reduced; if needed the reduction is temporarily held with pins. A guide pin is passed from the radial surface of the scaphoid, transversely through this entire carpal bone, across the scapholunate joint, and partially into the adjacent lunate. A cannulated drill is then extended along the guide pin to drill a 2 mm to 5 mm tunnel through the scaphoid, scapholunate joint, and partially into the interior of the lunate. Ideally, the guide pin should be inserted in the vicinity of the central axis of rotation of between the scaphoid and lunate. Preferably, the insertion site for the guide pin is nearly centered between the dorsal and palmar margin of the scaphoid.

Next, a guide, preferably E-shaped, is used to create a second hole entirely through the lunate, extending from the dorsal side to the palmar side, perpendicular to the first tunnel and intersecting the tunnel at its endpoint, to create a T-shaped channel within the lunate. This E-shaped guide can include a U-shaped member and central arm. The U-shaped member is preferably made of a radiolucent material to overcome the problem of obscuring the position of the central arm of the guide on X-ray, with collinear guide holes proximate the ends of opposing dorsal and palmar arms. The central arm includes a slotted eyelet which is collinear to the dorsal and palmar guide holes. This central arm preferably is made of a non-radiolucent material, facilitating the use of X-ray imaging to verify its proper positioning.

The E-shaped guide is positioned by inserting the central arm through the scaphoid, through the scapholunate joint, and into the lunate, and its accurate positioning is then confirmed by X-ray. First and second guide sleeves are then inserted into the opposing guide holes of the dorsal and palmar arms of the E-shaped guide, respectively. Next, a 2.0 mm drill is employed to create the second, vertical hole entirely through lunate, by extending the drill through the first guide sleeve, the dorsal arm, the slotted eyelet of the central arm, the palmar arm, and the second guide sleeve. Alternatively, the drill may be passed from palmar to dorsal.

A rigid pin and trailing tendon wire is next passed entirely through this second hole, by passing the pin and a portion of the tendon wire through all three collinear holes of the E-shaped guide. This pin and wire combination includes a rigid pin having one end swedged over or otherwise attached to a flexible wire, which may be manufactured from monofilament nylon (i.e., fishing line), braided wire, or nickel-titanium (nitinol) wire. Preferably, this tendon wire has a second rigid pin of smaller diameter swedged on or otherwise attached to the trailing portion of the flexible wire, and may have an optional wire loop attached to its trailing end. The guide sleeves are then removed, and the central arm of the E-shaped guide is withdrawn from the tunnel, pulling with it a looped, flexible central portion of the tendon wire out of the opening in the scaphoid.

Next, the tendon graft is looped about the tendon wire, and the ends of the tendon may be sutured together if desired. A pushing instrument is then used to guide the central loop of the tendon through the scaphoid, across the scapholunate joint and fully into the hole in the lunate. In a preferred embodiment, this pushing instrument is forked to guide the loop of the tendon graft into the hole. Alternatively, the two ends of the tendon wire are drawn apart, drawing the looped end of the graft through the scaphoid, scapholunate joint, and into the lunate. The tendon wire is withdrawn, pulling the larger diameter leading guide pin of the tendon wire back through the central loop of the graft in order to dilate it. Next, the tendon wire is advanced until the smaller diameter trailing guide pin engages the loop of the tendon graft; this smaller guide pin is sized to fit the central cannulation of the tendon screw.

A threaded, cannulated tendon screw is then extended over the tendon wire, through the dorsal opening of the lunate, through the loop of the graft, and towards the palmar opening in the lunate. In a preferred embodiment, the top and bottom portion of the screw are threaded with a smooth region therebetween in the area where the graft loop will be retained. Preferably, the bottom or leading thread is slightly smaller and rounder in order to avoid wrapping of the tendon by the screw as it is inserted. In another embodiment, only the top portion is threaded.

In another embodiment, a flexible line is passed through the second, vertical hole in the lunate. The central loop of this flexible line is withdrawn out the tunnel in the scaphoid and lunate and a tendon graft is looped through the loop of the flexible line. The graft is then positioned into the tunnel and the flexible line pulled taut. A cannulated guide pin is then threaded over the flexible line through the loop of the tendon graft. The threaded cannulated lunate screw is then directed over the guide pin, securing the loop of tendon in the lunate.

Next, the two arms of the graft, opposite the looped side, are pulled tightly away from the scaphoid, drawing the scaphoid and lunate together and shortening the gap between them. An optional interference screw or bone graft may be inserted if desired into the hole in the scaphoid to retain the graft between the sidewall of the hole and the screw. A bioabsorbable interference screw may alternatively be used to retain the graft at the scaphoid opening. Alternatively, no bone graft or interference screw is used but the graft is secured to the superficial surface of the scaphoid and/or lunate.

The tails of the graft extending out of the scaphoid hole are then looped around to the dorsal lip of the proximal scaphoid and secured in this region either with a suture anchor or with suture through drill holes in the bone. The remaining arm of the graft is then directed over the scapholunate joint, and on top of the dorsal surface of the lunate. These free arms are secured to the dorsal surface of the lunate. The graft may be secured with a suture that is placed through the cannulation of the lunate tendon screw and anchored on the palmar surface, with a suture extending through drill holes, in bone or with a suture anchor. This adds an additional layer of reconstruction and serves to further inhibit undesirable pronation and supination movement of the scapholunate joint. Optionally, the surgeon can add further reinforcement by continuing the graft over to the triquetrum and adding additional sutures or suture anchors to secure the end of the graft.

For this method, suture anchors in the general shape of screws, expandable anchors, buttons or beads may be used instead or in addition to affix an end of a suture proximate the lunate screw, towards securing the distal arms of the graft to the lunate. Alternatively, a looped end of the suture can be secured on the palmar surface of the lunate screw simply by tying a knot around the loop with a second heavier suture that is sized to be too large to pass through the cannulation in the screw.

### BRIEF DESCRIPTION OF THE SEVERAL VIEWS OF THE DRAWINGS

FIG. 1A of the drawings is a dorsal view of the carpal bones of the human hand, showing a dorsal view of the entire hand in an inset thereof;

FIG. 1B is a palmar view of the carpal bones of the human hand, showing a palmar view of the entire hand in an inset thereof;

FIG. 1C is a proximal view, looking distally, of the carpal bones of the human hand;

FIG. 2 is a simplified schematic, sectional view of the scaphoid and lunate and showing, in particular, an implanted lunate screw securing a looped end of an intramedullary tendon graft;

FIG. 3A is a perspective view of the E-shaped guide;

FIG. 3B is a rear view of the E-shaped guide;

FIG. 3C is a sectional view of the E-shaped guide, taken generally along lines 3C-3C of FIG. 3B;

FIG. 4A is a top perspective view of the guide pin drill guide and associated referencing arm;

FIG. 4B is a bottom perspective view of the guide pin drill guide and associated referencing arm;

FIG. 4C is a front view of the guide pin drill guide and associated referencing arm;

FIG. 5 is a front view of the cannulated drill;

FIG. 6A is a side view of the lunate screw;

FIG. 6B is a top plan view of the lunate screw;

FIG. 6C is a sectional view of the lunate screw, taken generally along lines 6C-6C of FIG. 6A;

FIG. 7A is a side view of the optional interference screw;

FIG. 7B is a top plan view of the optional interference screw;

FIG. 7C is a sectional view of the optional interference screw, taken generally along lines 7C-7C of FIG. 7A;

FIG. 8 is a perspective view of the graft pusher;

FIG. 9 is a top plan view of the scapholunate reconstruction system kit;

FIG. 10 is a simplified palmar view of the carpal bones of the human hand and showing, in particular, the reduction of the scapholunate gap;

FIG. 11 is a simplified palmar view of the carpal bones of the human hand and showing, in particular, the insertion and orientation of the guide pin from a palmar vantage point;

FIG. 12 is a simplified proximal view, looking distally, of the carpal bones of the human hand and showing, in particular, the insertion and orientation of the guide pin from a proximal vantage point.

FIG. 13 is a palmar view of the carpal bones of the human hand and showing, in particular, the drilling of the trans-scaphoid/lunate hole;

FIG. 14 is an X-ray of the E-shaped guide with the central arm positioned within the scaphoid and lunate and showing, in particular, use of the radiolucent nature of a portion of the E-shaped guide to confirm proper placement of the central arm within the trans-scaphoid/lunate hole;

FIG. 15 is a proximal view, looking distally, of the carpal bones of the human hand and showing, in particular, the insertion of the drill sleeves through associated apertures of the E-shaped guide;

FIG. 16 is a simplified a proximal view, looking distally, of the carpal bones of the human hand and a simplified schematic view of the E-shaped guide and associated drill sleeves and showing, in particular, the drilling of the vertical lunate hole;

FIG. 17 is a proximal view, looking distally, of the carpal bones of the human hand and showing, in particular, the placement of the tendon wire through the vertical lunate hole and through portions of the E-shaped guide;

FIG. 18 is a proximal view, looking distally, of the carpal bones of the human hand and showing, in particular, the removal of the central arm of the E-shaped guide from the trans-scaphoid/lunate hole to draw a loop of the tendon wire out of the scaphoid;

FIG. 19 is a proximal view, looking distally, of the carpal bones of the human hand and showing, in particular, the looping of the tendon graft through a loop of the tendon wire;

FIG. 20 s a proximal view, looking distally, of the carpal bones of the human hand and showing, in particular, the loop of the tendon graft seated within the lunate;

FIG. 21 is a simplified schematic, sectional view of a combination of the scaphoid and lunate and showing, in particular, the use of the graft pusher to insert the tendon graft into the scaphoid and lunate;

FIG. 22 is a proximal view, looking distally, of the carpal bones of the human hand and showing, in particular, the insertion of the lunate screw;

FIG. 23 is a proximal view, looking distally, of the carpal bones of the human hand and showing, in particular, the dorsal reflection of the distal arms of the tendon graft;

FIG. 24A is a side view of a suture loop for securing the distal arms of the tendon graft;

FIG. 24B is a side view of the suture loop of FIG. 8A and showing, in particular, the preparation of the suture loop for placement about the button-shaped suture anchor;

FIG. 24C is a side view of the button-shaped suture anchor;

FIG. 24D is a side view of the suture loop secured to the button-shaped suture anchor using a Lark's Head knot;

FIG. 25A is a side view of suture loop in preparation for insertion through the bead-shaped suture anchor;

FIG. 25B is a side view of the suture loop and showing, in particular, the preparation of the suture loop for placement about the bead-shaped suture anchor;

FIG. 25C is a side view of the suture loop secured to the bead-shaped suture anchor using a Lark's Head knot; and

FIG. 26A is a side view of another embodiment of a suture anchor;

FIG. 26B is a side view of yet another embodiment of a suture anchor; and

FIG. 27 is a proximal view, looking distally, of the carpal bones of the human hand and showing, in particular, the dorsal reflection of the distal arms of the tendon graft.

### DETAILED DESCRIPTION OF THE INVENTION

While several different embodiments of the present invention are described herein and shown in the various figures, common reference numerals in the figures denote similar or analogous elements or structure amongst the various embodiments.

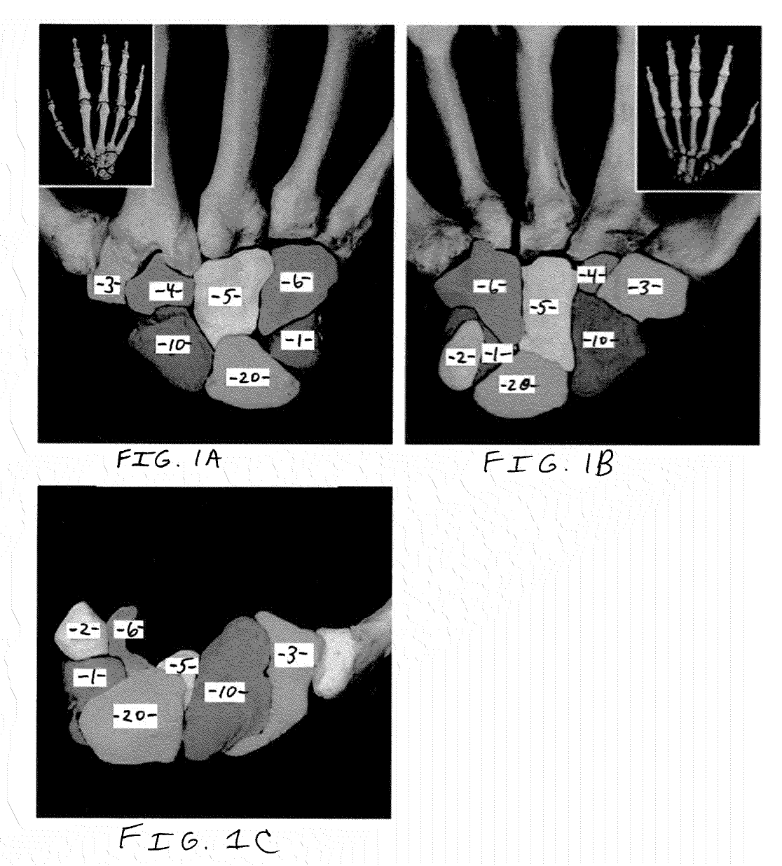

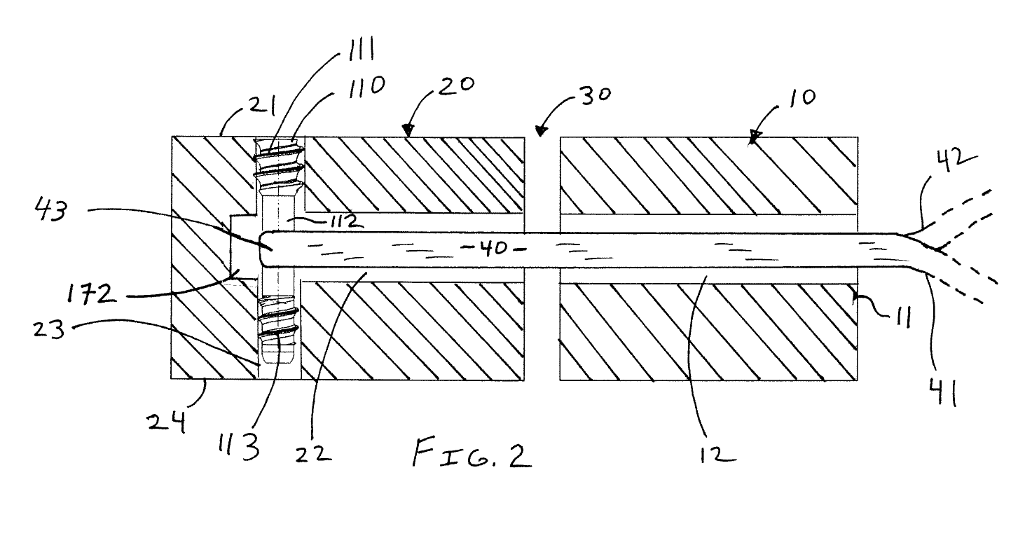

The carpal bones of a human right hand are shown in FIGS. 1A-1C as comprising the triquetrum 1, pisiform 2, trapezium 3, trapezoid 4, capitate 5, hamate 6, scaphoid 10 and lunate 20. One aspect of the current invention is a method, and associated instruments, for performing scapholunate ligament reconstruction—i.e., repairing injury to the group of scapholunate interosseous ligaments, proximate the scapholunate joint.

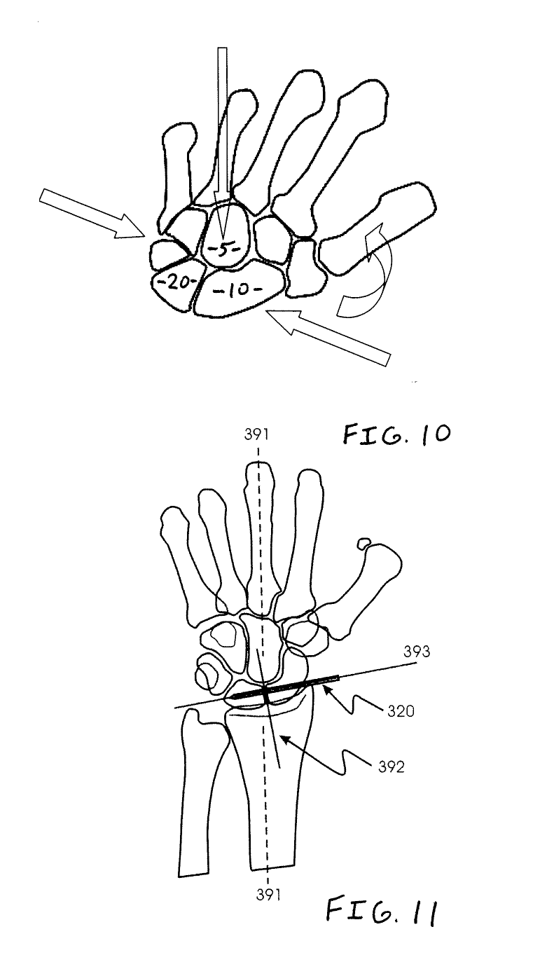

The methods of the present invention perform ligament reconstruction using a tendon graft to create a replacement for the function of the scapholunate ligaments. The graft is placed in the vicinity of the center axis of rotation at the base of the scaphoid. The result of this reconstruction is shown in a simplified, schematic form in FIG. 2. A suitable tendon graft 40 of the palmar longus or other similar tendon and of suitable length is harvested, using tendon stripper 310 of scapholunate reconstruction system kit 300, shown in FIG. 9.

Next, exposure of the carpus is done as needed. Typically, this will include dorsal exposure of the carpus with a skin incision radial to Lister's tubercle. A skin flap is elevated radially to an interval between the first and second compartment distal to the tip of the styloid, with the styloid tip being removed, if necessary. A skin flap is also elevated ulnarly to the triquetrum. The fourth compartment retinaculum is typically incised and, from within the fourth compartment, the radial septum is incised to the third compartment. The dorsal wrist capsule is typically incised transversely from the radial border of the second compartment to the mid-portion of the dorsal radiocarpal ligament, continuing obliquely to triquetrum.

Following exposure, the scapholunate joint is reduced, as shown in FIG. 10. In particular, as shown by the arrows of FIG. 10, scaphoid 10 is supinated to correct pronation, the capitate is pushed palmarly, and the scapholunate gap is closed. Specifically, the wrist is placed in approximately 20° of ulnar deviation, to expose the entry site of scaphoid 10. Capitate 5 is translated palmarly to over correct lunate 20 flexion as much as possible. A heavy pin is placed from the ulnar side of the dorsal radius through the radiocarpal joint to engage and stabilize lunate 20. This pin should be placed ulnarly to avoid instrumentation in the rest of the present procedure. If necessary, a tenaculum may be employed to close the scapholunate gap and pin scaphoid 10 to capitate 5 in a manner distal enough to avoid instrumentation in the rest of the procedure.

Next, as shown in FIGS. 11 and 12, a guide pin, such as Kirschner or K-wire 320, is passed from radial surface 11 of scaphoid 10, transversely across scaphoid 10, across scapholunate joint 30 and partially into lunate 20. The inventor has observed that the normal axis of rotation of the scaphoid in relation to the lunate is along an axis that is directed into the central part of the lunate between the dorsal/palmar surfaces, and is positioned as close to the distal surface of the lunate as possible. This corresponds to the center of a circle that is drawn around the base of the lunate and appears to be the primary transverse rotation axis of the scaphoid.

Guide pin 320 is preferably positioned along an axis 393 that is normal to the plane of the scapholunate joint 392 as shown in FIG. 11. In general, when the hand is in neutral position with the third metacarpal aligned with the long axis of the forearm 391, the plane of the scapholunate joint 392 is inclined approximately 17 degrees to axis 391. Moreover, as shown by dashed 391 coordinate axis of FIG. 12, guide pin 320 is preferably positioned at an angle of approximately five degrees supination.

A point of entry is selected for the guide pin that is approximately halfway between the dorsal/palmar surface of the scaphoid. The guide pin is inserted along the rotational center of the scaphoid (relative to the lunate), and across the scapholunate joint. In general, the guide pin should enter the lunate near its distal margin and is inclined proximally towards the triquetrum.

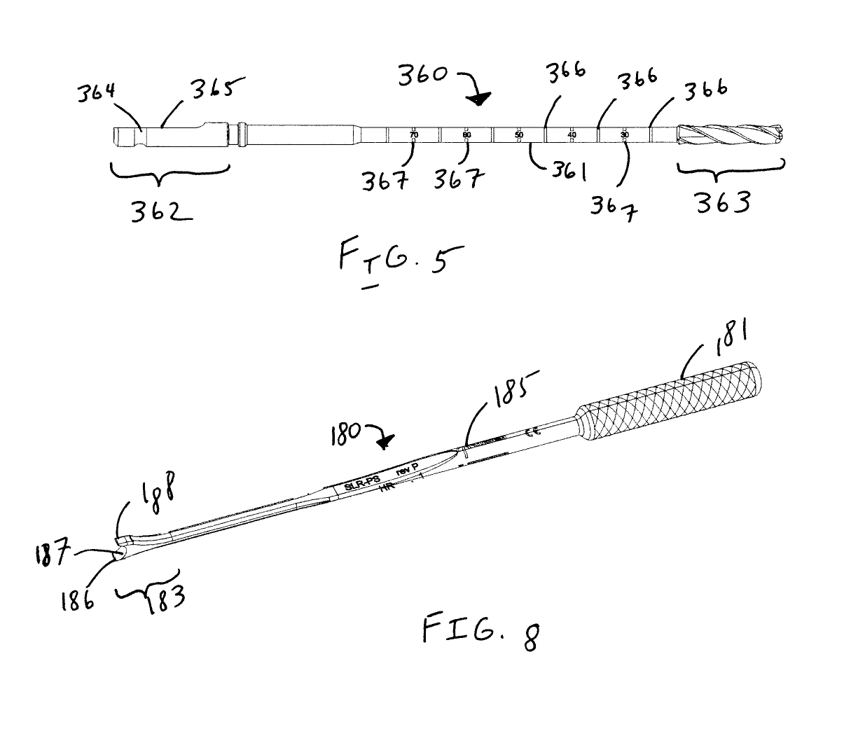

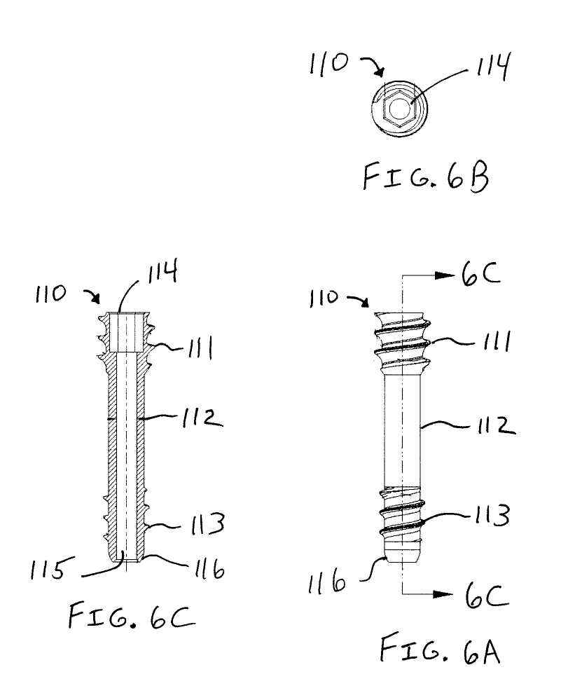

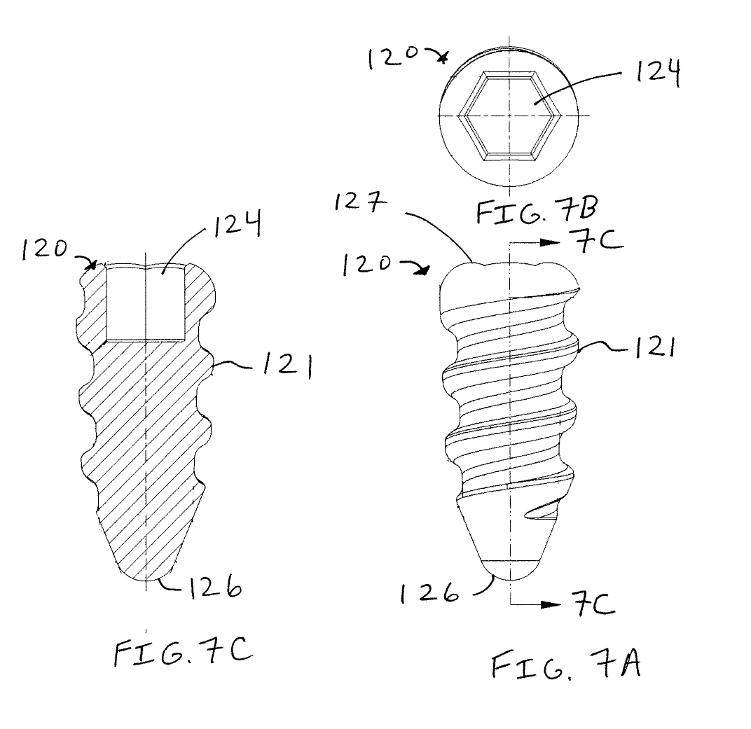

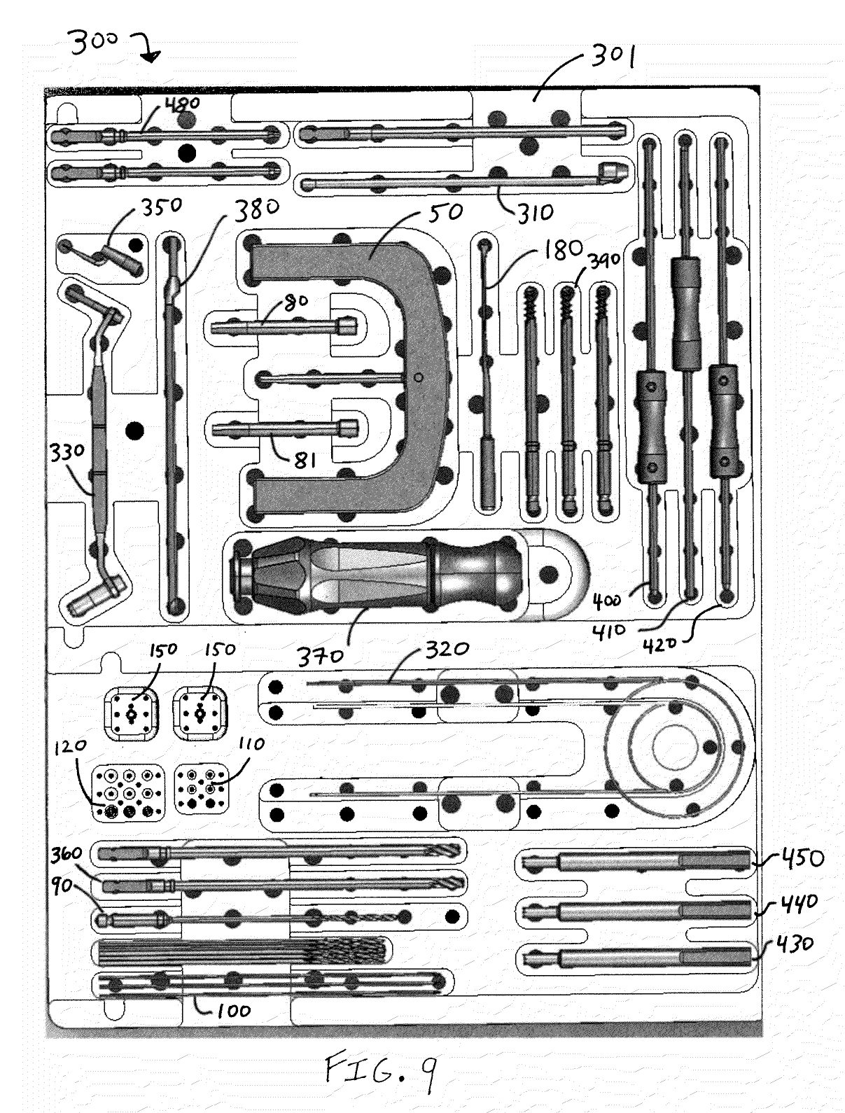

Placement of guide pin 320 may be facilitated with the use of guide pin drill guide 330, shown in FIGS. 4A through 4C as comprising elongated central body 331, first arm 332, and second arm 333 at opposing ends of central body 331. First arm 332 includes an aperture through which first drill guide sleeve 334 is inserted, and second arm 333 includes an aperture through which second drill guide sleeve 335 is inserted. In an embodiment of the present invention, first drill guide sleeve 334 includes a channel extending from top aperture 336 to bottom aperture 338 of approximately 1.6 millimeters in diameter. Second guide sleeve 335 includes a channel extending from top aperture 337 to bottom aperture 339 of approximately 4.0 millimeters in diameter, sized to accommodate a 3.6 millimeter cannulated drill. Bottom apertures 338 and 339 both have chamfered and serrated openings, serving to inhibit unwanted slippage of the drill guide sleeve upon placement adjacent a targeted region of bone. As shown in FIG. 4C, first arm 332 and second arm 333 are each set at an angle 340 of approximately thirty degrees, relative to longitudinal axis 341 of elongated central body 331.

As shown in FIGS. 4A through 4C, drill guide locator sleeve 350 is releasably attachable to first drill guide sleeve 334. The use of a detachable drill guide locator sleeve permits the option of selecting from amongst a plurality of locator sleeves having referencing arms of different, predetermined sizes to center the insertion site in order to accommodate a range of differently sized scaphoids that may be encountered. Preferably, a selected referencing arm places the entry site approximately five millimeters dorsal to the palmar surface of the proximal scaphoid.

Drill guide locator sleeve 350 includes cylindrical top portion 351 having top aperture 352 and four vertical slots 353, equally spaced at ninety degree intervals about the circumference of top portion 351 and permitting top portion 351 to flex to facilitate secure yet releasable attachment of drill guide locator sleeve 350 about first drill guide sleeve 334. Drill guide locator sleeve 350 further includes referencing arm 354, having an upper curved region 355 proximate cylindrical top portion 351.

In an embodiment of the present invention, a drill guide locator sleeve 350 is provided wherein cylindrical top portion 351 and referencing arm 354 are each approximately 0.500 millimeters in length, and curved region 354 provides an offset of approximately 0.197 inches of referencing arm 354 relative to a central longitudinal axis of cylindrical top portion 351.

With drill guide locator sleeve 350 attached to first drill guide sleeve 334, referencing arm 354 is placed under the scaphoid, palmarly. This serves to position guide pin 320 approximately 5 millimeters from the volar surface, and aligns guide pin 320 as it is advanced through first drill guide sleeve 334 in order to center the entry site of guide pin 320 as it is further advanced between the dorsal and palmar surfaces of the proximal pole of the scaphoid.

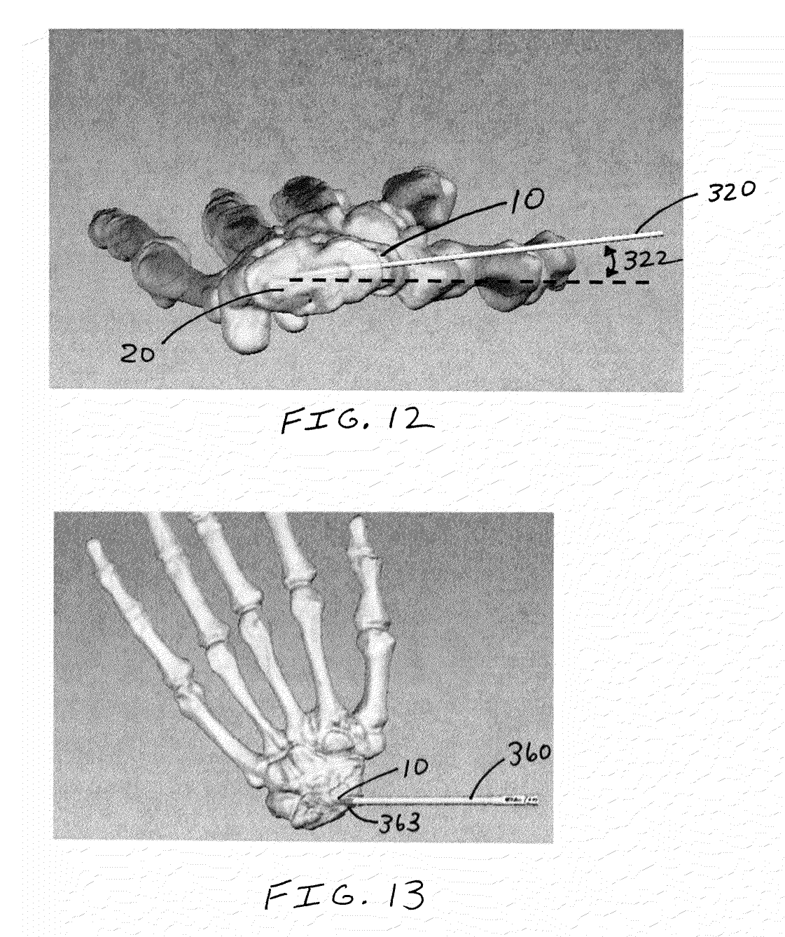

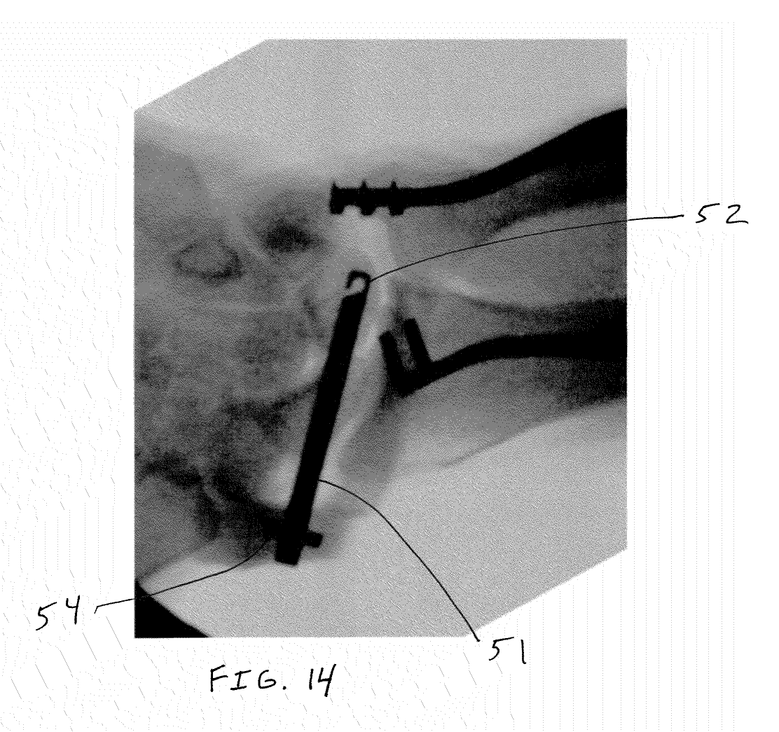

Next, as shown in FIG. 13, a cannulated drill bit, such as drill bit 360 of FIG. 5, is then drilled over guide pin 320, through scaphoid 10, across scapholunate joint 30 and partially into lunate 20, creating tunnel 12 through scaphoid 10 and tunnel 22, partially through lunate 20. Drill bit 360 is shown in FIG. 5 as comprising elongated shaft 361 having proximal handle attachment region 362 and distal cutting region 363. Proximal handle attachment region 362 includes ball detent 364 and planar surface portion 365, facilitating attachment of cannulated drill bit 360 to a “quick-change” type of drill. Elongated shaft 361 includes a plurality of graduated drill depth markings, including those 366 with bands only alternating with those 367 having both bands and numeric indicia. While a 3.6 mm cannulated drill is typically employed, any drill size between 3 mm and 5 mm may be used.

Prior to drilling, a depth gauge, such as pin depth gauge 380 of FIG. 9, is preferably employed to measure the depth of insertion of guide pin 320 to ascertain the required depth of the transverse scapholunate hole, using a second guide pin alongside to further confirm the required depth, and using a C-arm to check the depth of drill 360. The targeted position of the drill hole in lunate 20 is slightly over halfway between the scapholunate and lunotriquetral joints. The direction of the scapholunate hole tends to angle proximally in lunate 20 as it approaches the lunotriquetral joint. If the transverse hole is drilled too deeply in lunate 20 (i.e., too close to lunotriquetral joint), the vertical hole will end up too far proximal/ulnar on lunate 20.

If an optional interference screw, such as interference screw 120 of FIGS. 7A through 7C, is to be employed in securing the graft to the scaphoid, a tap, such as 4.0 millimeter tap 390 of FIG. 9, may be employed to prepare the entry region of scaphoid hole to receive optional interference screw 120.

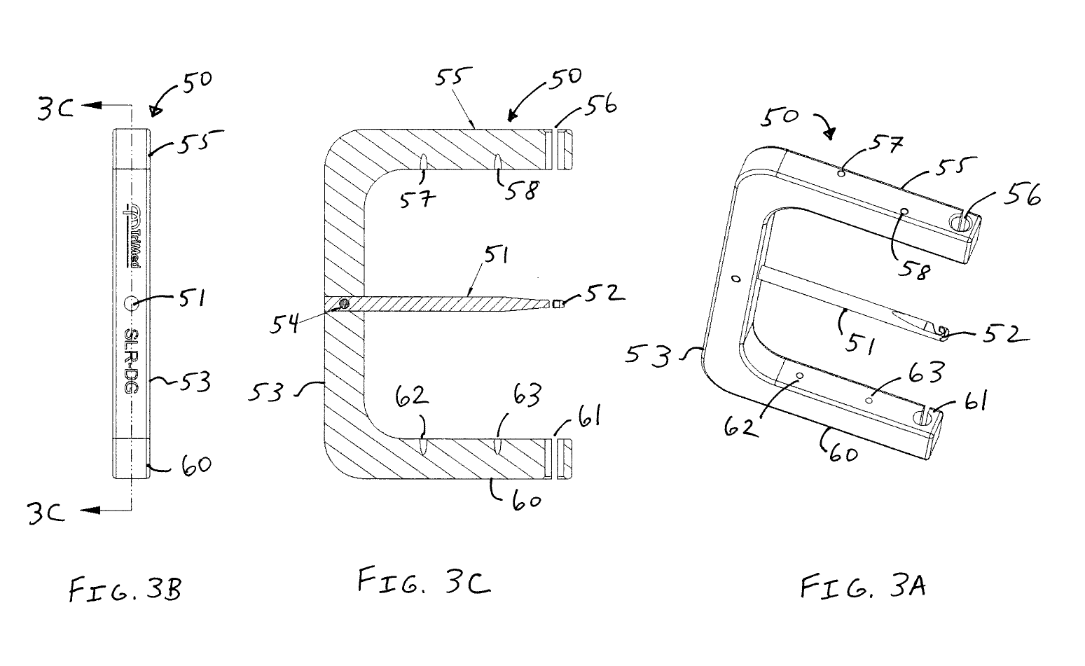

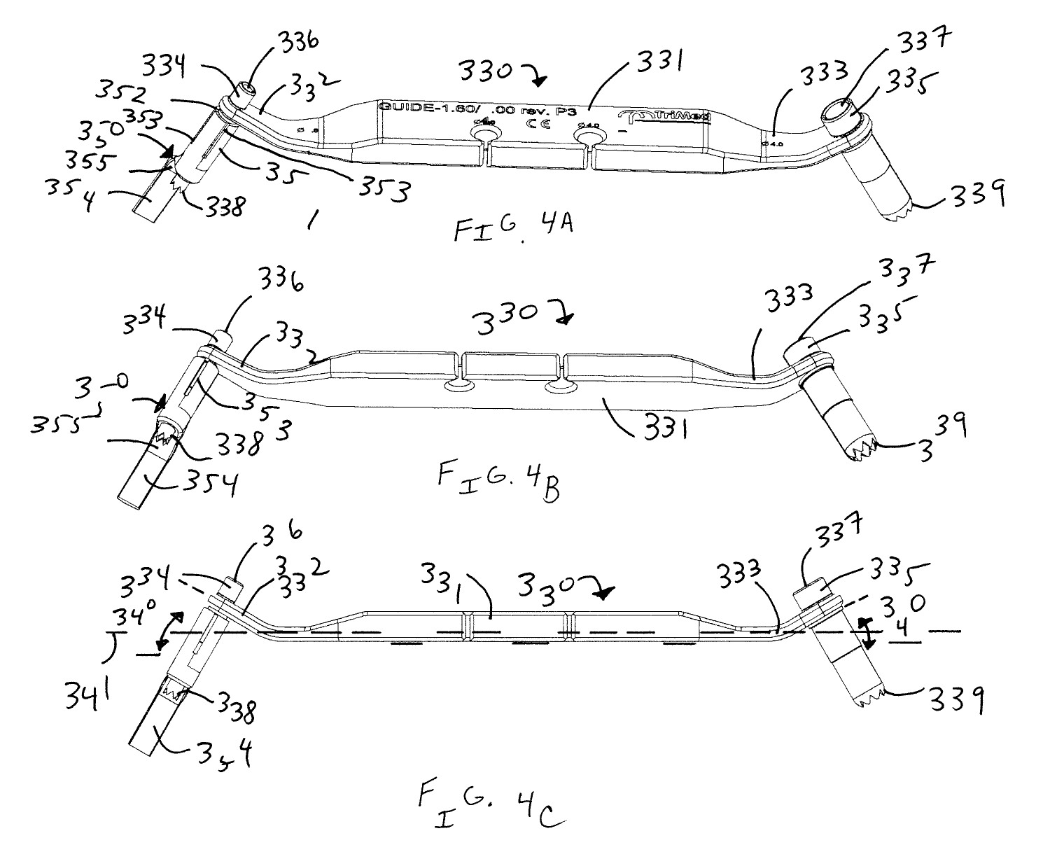

Once tunnels 12 and 22 are drilled through scaphoid 10, across the scapholunate articulation, and partially into lunate 20, a guide, such as E-shaped guide 50, shown in FIGS. 3A through 3C, is then used to position a secondary, vertical lunate hole 23 (as shown in FIG. 2) to be drilled through lunate 20, intersecting and orthogonal to tunnel 22 proximate an endpoint of tunnel 22 (though, as shown in FIG. 2, tunnel 22 may extend beyond hole 23 to include region 172 to facilitate graft placement), extending from dorsal side 21 to palmar side 24 of lunate 20.

Referring to FIGS. 3A-3C, E-shaped guide 50 includes central arm 51 that is sized for insertion into the scapholunate tunnel comprising substantially collinear scaphoid tunnel 12 and lunate tunnel 22. Central arm 51 has a central end with a substantially hook-shaped opening 52 creating an associated eyelet, and may be attached to U-shaped member 53 by cross pin 54. U-shaped member 53 includes dorsal arm 55 and palmar arm 60. Dorsal arm 55 includes slotted dorsal aperture 56. Palmar arm 60 includes slotted palmar aperture 61. As best seen in FIG. 3C, slotted dorsal aperture 56, hook-shaped opening 52, and palmar aperture 61 are substantially collinear to each other. E-shaped guide 50 may optionally include cross-angled apertures 57, 58, 62 and 63 extending through dorsal arm 55 and palmar arm 60, permitting the insertion of K-wires therethough to assist in maintaining E-shaped guide 50 in position as lunate hole 23 is drilled.

Central arm 51 is inserted through scaphoid tunnel 12 and into lunate tunnel 22, until hooked end 52 of central arm 51 is proximate or abuts an endpoint of lunate tunnel 22. Slotted dorsal aperture 56 of dorsal arm 55 is preferably positioned proximate the center or just past the radial/ulnar mid-line of the lunate from the anterior posterior view. Placement too far ulnar may result in placement of a lunate screw too proximal in the lunate. The position of the volar arm of E-shaped guide 50 is used to make a volar incision, ulnar to the median nerve. Dissection is performed bluntly to the palmar capsule. Once so positioned, dorsal arm 55 and palmar arm 60 of E-shaped guide 50 are disposed above and below the wrist, permitting secondary lunate hole 23 be created from palmar side 24 to dorsal side 21 of lunate 20 (or dorsal to palmar).

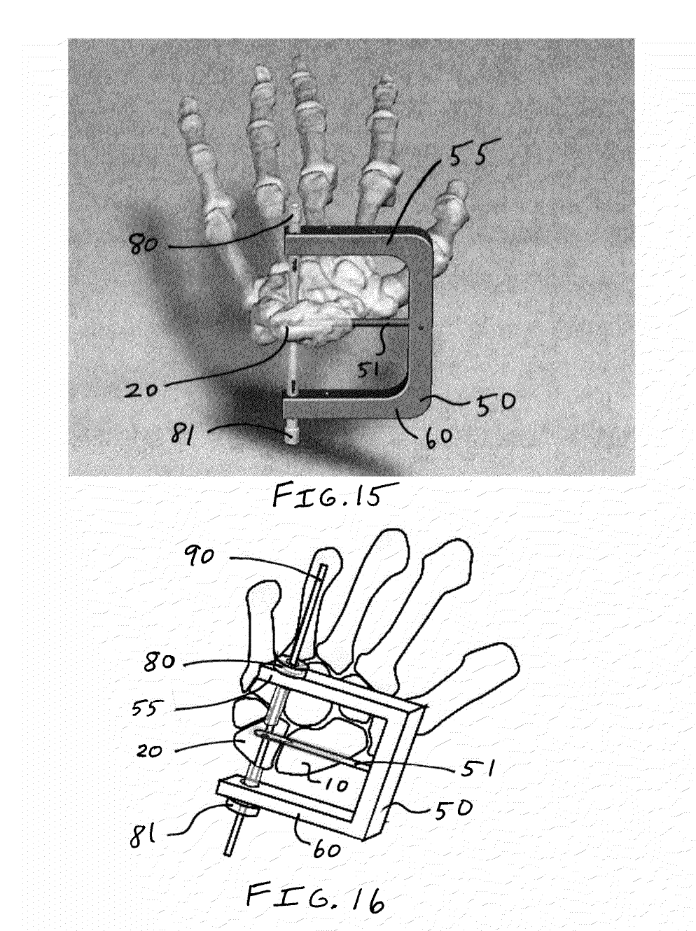

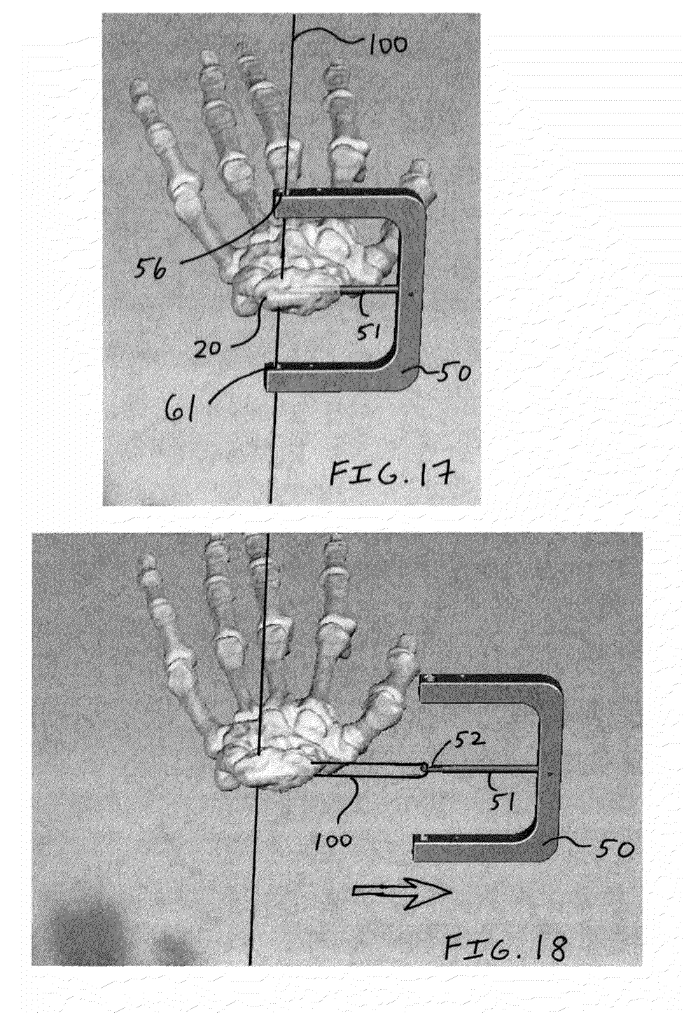

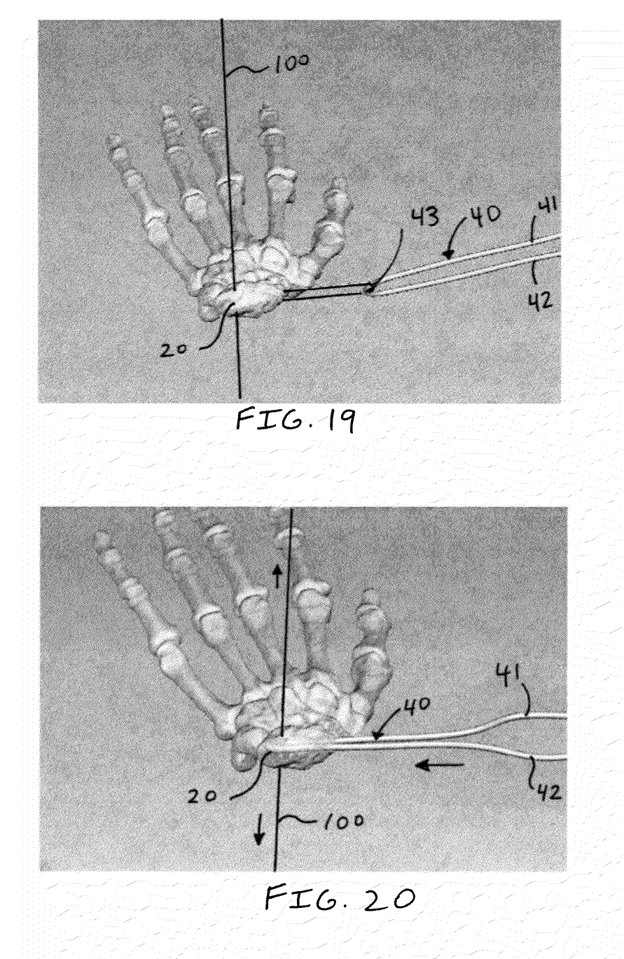

Once central arm 51 of E-shaped guide is inserted through scaphoid tunnel 12 and positioned within lunate tunnel 22, the accurate desired positioning of central arm 51 is preferably confirmed via X-ray. As demonstrated in FIG. 14, U-shaped member 53 of E-shaped guide 50 is preferably constructed of a substantially radiolucent material, leaving only central arm 51 and cross pin 54 visible in the X-ray, facilitating the determination of the accurate positioning of central arm 51.

Next, referring to FIG. 15, dorsal drill guide sleeve 80 is then inserted through slotted dorsal aperture 56 of E-shaped guide 50 until seated against the dorsal surface of lunate 20, and an identically configured palmar drill guide sleeve 81 is inserted through slotted palmar aperture 61 until seated against the palmar surface of the lunate. Each guide sleeve 80, 81 is sized to form a friction fit with its associated aperture 56, 61 of E-shaped guide 50.

Next, referring to FIG. 16, as drill guide sleeves 80 and 81 are held against lunate 20, a drill, such as 2.0 mm drill 90, is then placed from palmar to dorsal, or alternatively from dorsal to palmar, passing through guide sleeve 80, through slotted dorsal aperture 56, the aperture of hooked end 52 of central arm 51, and exiting through palmar guide sleeve 81 and slotted palmar aperture 61. Palmar arm 60 of E-shaped guide 50, in conjunction with palmar guide sleeve 81, ensures that drill 90 does not damage or sever the important nerves, arteries, and tendons on the palmar side as the tip of drill 90 exits palmar side 24 of lunate 20. Moreover, drilling from palmar to dorsal is preferred, as the drill guide sleeve ensures that important nerves, arteries, and tendons are not wrapped or injured by the drill, and drilling in this direction lessens the risk of drilling the median nerve, should drill 90 somehow to miss the far drill guide sleeve upon exit.

Since lunate 20 is relatively small in size, the use of a two-armed drill guide and associated sleeves serves to satisfy the significant physical accuracy constraints imposed by the small size of lunate 20, relative to the placement of transverse secondary lunate hole 23. However, a substantially C-shaped, single-armed drill guide, having a central arm and a single dorsal or palmar outer arm, of similar construction to the dorsal and palmar arms of E-shaped guide 50, may alternatively be employed.

Next, as shown in FIG. 17, once drill 90 has been placed, it is removed and flexible guide wire 100 is placed through the newly formed vertical secondary lunate hole 23 which, together with lunate tunnel 22, forms a substantially T-shaped intramedullary channel within lunate 20. Several different embodiments of the tendon wire 100 are contemplated. In most of the contemplated embodiments, a length of flexible material is attached to a rigid pin by swedging or otherwise attaching the flexible material onto the pin. The rigid pin is constructed of a material stiff enough to enable it to easily pass through dorsal guide sleeve 80, through the eyelet of hooked end 52 of central arm 51 of E-shaped guide 50, and out of palmar guide sleeve 81. Meanwhile, the flexible material must be flexible enough to allow a loop of the flexible material to be pulled out through lunate tunnel 22 and scaphoid tunnel 12, as will be described in detail, infra. Moreover, both the rigid pin and the flexible material must be small enough to pass through the 2 mm diameter of secondary lunate hole 23.

In a first embodiment of the tendon wire 100, a length of monofilament nylon is attached to a trailing end of the rigid pin. In a second embodiment of the tendon wire 100, a length of braided wire is attached to a trailing end of the rigid pin. In a third embodiment of the tendon wire 100, a length of a NITINOL (NIckel TItanium Naval Ordnance Laboratory) wire, a relatively stiff wire that can be bent but retains a ‘memory’ and straightens out spontaneously when the bending for is released, is attached to a trailing end of the rigid pin. In another embodiment, the tendon wire is simply a flexible piece of cable, wire or suture. In this embodiment, a stiff pin with a central cannulation is then thread over the flexible wire to guide the insertion of the lunate screw. In another embodiment, the tendon wire is of a form of two guide pins that are connected by an intermediate flexible wire, cable, or the like. The leading pin is of a diameter that is large enough to go through the vertical hole in the lunate and can be used to dilate the loop of tendon graft when it is in place. The trailing pin is sized to pass through the cannulation in the lunate screw in order to direct the screw through the graft loop after it has been dilated. In addition, a small trailing loop of wire or suture at the back end of the second pin allows a loop of suture to be shuttled through the center cannulation of the lunate screw as a means of anchoring the suture.

The leading rigid pin portion of the tendon wire 100 is passed through dorsal guide sleeve 80 and slotted dorsal aperture 56, into secondary lunate hole 23, through the eyelet of hooked end 52 of central arm 51, further across secondary lunate hole 23, through palmar guide sleeve 81 and slotted palmar aperture 61, and out of palmar surface 24 of lunate 20. The rigid pin is pulled completely out of lunate 20, leaving the trailing wire traversing the entirety of transverse secondary hole 23. Of course, alternatively, the tendon wire can be passed from palmar to dorsal.

Next, as shown in FIG. 17, dorsal guide sleeve 80 and palmar guide sleeve 81 are removed by sliding them in opposing directions along tendon wire 100. Tendon wire 100 is then uncoupled from dorsal arm 55 and palmar arm 60 of E-shaped guide 50 by pulling portions of the tendon wire 100 through the slots of slotted dorsal aperture 56 and slotted palmar aperture 61.

Referring to FIG. 18, central arm 51 of E-shaped guide 50 is then pulled out of lunate tunnel 22 and scaphoid tunnel 12, bringing with it a central portion of the tendon wire 100 that is engaged by hooked end 52 of central arm 51. Next, as shown in FIG. 19, central portion 43 of graft 40 is then looped around the central loop of the tendon wire 100, and a strong running suture may optionally be used to sew distal arms 41, 42 of graft 40 together, starting approximately five millimeters away from the central loop of the tendon wire 100 and continuing down toward the distal free end arms 41 and 42 of graft 40.

Next, as shown in FIG. 20, the two free ends of tendon wire 100, exiting the distal and palmar openings of vertical secondary hole 23, respectively, are pulled apart from each other until the portion of tendon wire 100 therebetween becomes taut. This, in turn, pulls the central loop of tendon wire 100, together with the looped end 43 of graft 40 looped about the central loop of tendon wire 100, into the opening of scaphoid tunnel 12 adjacent radial surface 11 of scaphoid 10, through the entirety of scaphoid tunnel 12, across scapholunate articulation, or joint 30, and into lunate tunnel 22, until looped end 43 of graft 40 reaches the juncture of lunate tunnel 22 and vertical secondary lunate hole 23. Optionally, tendon wire 100 is then withdrawn until the larger pin is drawn back up into the vertical lunate hole 22, through the loop 43 in graft 40 and out the top of the lunate 20, in order to dilate the opening of the loop 43 in graft 40 and ensure that it is not adherent to tendon wire 100. Optionally, tendon wire 100 may be pulled back and forth through secondary lunate hole 23 several times in an oscillating manner, to ensure that graft 40 slides freely over tendon wire 100 and that looped end 43 is completed seated in transverse lunate hole 23. Tendon wire 100 is then advanced until its trailing pin is seated fully in vertical hole 23 in lunate 20, including passing through loop 43 in tendon graft 40.

Alternative methods of positioning and seating looped end 43 of graft 40 at the junction of lunate tunnel 22 and vertical secondary lunate hole 23 are also contemplated. For example, in FIG. 21, scaphoid 10, lunate 20, and scapholunate articulation 30 are shown in combined, simplified schematic form, viewed from the dorsal side, and are identified by reference numeral 170. Moreover, scaphoid tunnel 12 and lunate tunnel 22 are shown in combined, simplified schematic form, viewed from the dorsal side and with the scapholunate gap omitted, and are identified by reference numeral 171.

In this alternative method, the scaphoid and lunate tunnels, as well as vertical lunate hole 23, are prepared in the manner previously described. Moreover, in the manner previously described, tendon wire 100 is passed through lunate hole 23 and hooked end 52 of central arm 51 of E-shaped guide 50, and central arm 51 is withdrawn from the scaphoid and lunate tunnels, pulling a central loop of the tendon wire 100 out of the radial side of the scaphoid. Furthermore, in the manner previously described, graft 40 is looped around the central loop of tendon wire 100 to form graft loop 43, and distal arms 41 and 42 of graft 40 may be sewn together, preferably with a strong running suture. Next, as shown in FIG. 21, a forked implement, such as graft pusher 180, shown in simplified form in this figure, is employed to engage graft loop 43, and to push graft loop 43 through the scaphoid and lunate tunnels 171 until graft loop 43 is seated at or beyond the junction of the scaphoid and lunate tunnels 171 and transverse lunate hole 23. Graft pusher 180 includes handle region 181, elongated shaft 182, and forked end 183, having two tines. Looped end 43 of graft 40 is engaged between the tines of graft pusher 180, and is then pushed by graft pusher 180 fully through the scaphoid tunnel until it is fully seated at the end of the lunate tunnel, proximate the juncture of the lunate tunnel with transverse secondary lunate hole 23. Next, graft pusher 180 is removed from the scaphoid and lunate tunnels 171, leaving looped end 43 of graft 40 in place. The use of graft pusher 180 is considered to be a more gentle method of seating graft 40 within lunate 20, reducing the risk of abrasion or other damage to graft 40.

Graft pusher 180 is shown in further detail in FIG. 8 as comprising elongated shaft 185, having a proximal end including handle 181, and a distal, forked end 183 including tines 186 and 188, separated by concave region 187, providing a smooth, arcuate surface for engaging looped end 43 of graft 40.

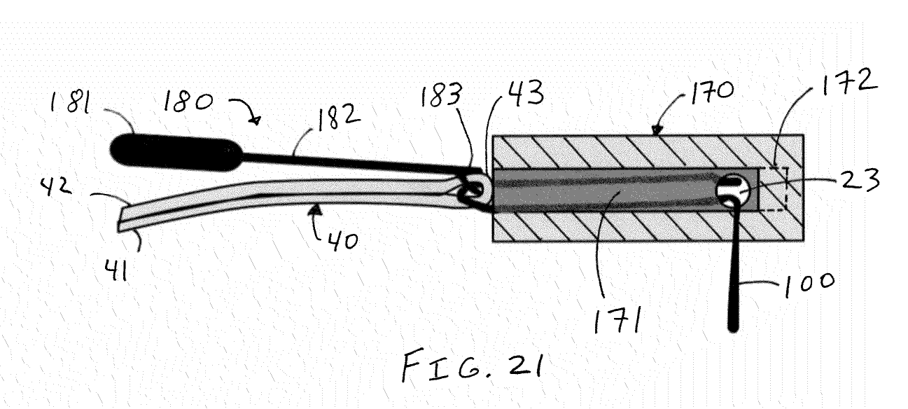

In a variation of this alternative method of positioning and seating looped end 43 of graft 40 at the junction of lunate tunnel 22 and vertical secondary lunate hole 23, vertical lunate hole 23 is not formed at the very end of lunate tunnel 22, but is instead formed to intersect lunate tunnel somewhat towards the radial side of its internal endpoint, further towards scapholunate articulation 30. This, in turn, creates an extended region 172 of the lunate tunnel, as shown in FIGS. 2 and 21. Referring to FIG. 21, in this variation, forked implement 180 is used to push looped end 43 of graft 40 completely into extended region 172 of the lunate tunnel, beyond the intersection of the lunate tunnel with transverse secondary lunate hole 23 so as to prevent wrapping of the loop of the graft around the tip of the lunate screw as it is advanced into the hole. Next, as shown in FIG. 22, regardless of how looped end 43 of graft 40 is seated within lunate 20, cannulated lunate screw 110 is then passed over the end of the trailing pin of the tendon wire 100 extending out of the dorsal side aperture of vertical lunate hole 23, and is then threaded down into lunate hole 23, typically using a cannulated hexagonal screwdriver, such as hex driver 480 in combination with handle 370 of FIG. 9. Alternatively, a cnanulated guide pin may be threaded over a simple flexible tendon wire 100 to guide cannulated screw 110. Tendon wire 100 thus guides cannulated lunate screw 110 down through vertical lunate hole 23, and through looped end 43 of graft 40, fixing looped end 43 in place, intramedullary to lunate 20 and disposed about smooth central region 112 of cannulated lunate screw 110. Once lunate screw 110 has been so placed, the distal end of graft 40 is tugged away from the scaphoid hole to insure graft 40 is well secured by lunate screw 110. Lunate screw 110 may be made out of any biocompatible material, such as metal or metal alloy, plastic, ceramics, or bioaborbable material.

A first embodiment of cannulated lunate screw 110 is shown in FIGS. 6A through 6C as comprising a generally cylindrical body having first threaded region 111 for threadably engaging lunate hole 23 proximate dorsal side 21, substantially smooth central region 112 for engaging looped end 43 of graft 40, and second threaded region 113 for threadably engaging lunate hole 23 proximate palmar side 24. Tapered end 116 facilitates the passage of cannulated lunate screw 110 through looped end 43 of graft 40, reducing the likelihood that the graft will be damaged by the passage of the lunate screw therethough. Tapered end 116 may be either substantially conical in shape, as shown in FIGS. 6A and 6C, or substantially hemispherical in shape. Elongated channel 115 communicates with hexagonal driver accepting region 114 to provide a contiguous passage extending through the entire length of cannulated lunate screw 110. In one embodiment, threaded region 113 is smaller in diameter than threaded region 111 and may include threads that are not as sharp to reduce the likelihood of damaging the graft as the screw is passed.

In another embodiment of cannulated lunate screw 110, second threaded region 113 is omitted, and substantially smooth central region 112 extends from first threaded region 111 to tapered end 116. The omission of second threaded region 113 eliminates the possibility of a threaded region of cannulated lunate screw 110 damaging looped end 43 of graft 40 as cannulated lunate screw 110 is guided along tendon wire 100 and through the aperture of looped end 43. In either embodiment of cannulated lunate screw 110, other configurations of driver accepting region 114, such as configurations accepting Phillips or TORX drivers, rather than hexagonal drivers, may alternatively be used.

In another embodiment of the present invention, a retaining member in the form of a cannulated screw is not employed. Instead, a retaining member in the form of a cannulated, generally cylindrical peg, having a plurality of ridges extending outwardly from the arcuate outer surface of the peg, is provided, and may be hammered into place, dorsally to palmary, within transverse secondary lunate hole 23.

Once cannulated lunate screw 110 has been threaded dorsally to palmary into transverse secondary lunate hole 23, loop 43 of graft 40 is fixed rigidly within lunate 20. Next, distal arms 41 and 42 of graft 40 are pulled tightly out of radial end 11 of scaphoid 10, closing the gap between scaphoid 10 and lunate 20 at scapholunate articulation 30.

In one embodiment, fixation of the tendon graft 40 within the scaphoid tunnel 12 is done with an interference screw 120 which is then inserted into a previously tapped region of scaphoid tunnel 12 at radial end 11, alongside graft 40, fixing a portion of graft 40 in place against the sidewall of scaphoid tunnel 12. As a result of the foregoing method, a relatively large tendon graft 40 is rigidly secured in both the scaphoid 10 and the lunate 20. The gap 30 between scaphoid 10 and lunate 20 is securely closed and dorsal migration of scaphoid 10 in relation to lunate 20 is prevented by graft 40, since it is disposed transversely across scapholunate joint 30. Furthermore, since graft 40 is flexible in a manner similar to a normal ligament, relative flexing or movement between scaphoid 10 and lunate 20 is permitted, without significant risk of hardware failure, loss of fixation, or bone destruction of the scaphoid, as may occur in a prior art RASL procedure. Use of an interference screw is not necessarily a preferred method if securing the distal arms of the tendon graft, however, due to concerns of fracturing the scaphoid as a result of hoop stress.

Sounds, such as plug pins 400, 410 and 420 of FIG. 9, may be used to confirm that sufficient room exists for placement of an interference screw of a desired size. In a preferred embodiment, plug pins 400, 410 and 420 are 3.0 mm, 3.4 mm and 3.8 mm in size, respectively. Plug pin 410, for example, ensures sufficient room for a 3.5 mm interference screw.

Interference screw 120 is shown in FIGS. 7A through 7C as comprising a substantially cylindrical body having threaded region 121, tapered end 126, and hexagonal driver accepting region 124. Other configurations of driver accepting region 124, such as configurations accepting Phillips or TORX drivers, rather than hexagonal drivers, may alternatively be used.

Interference screw 120 is preferably provided with a relatively rounded head region 127, as it is placed through a hole that enters a relatively oblique area of scaphoid 10. In one embodiment of the present invention, interference screw 120 is constructed of a bio-absorbable material. In another embodiment of the present invention, interference screw is constructed of PEEK (polyether-etherketone), a relatively hard, radiolucent non-absorbable plastic. Alternatively, the portion of the graft in the scaphoid hole 12 may be secured with osseous sutures, suture anchors, plugs, bone graft or bone dowels, or similar options well known in the art.

Another method of graft fixation via scaphoid interference is the use of a bone plug. Again, plug pins 400, 410 and 420 may be used as sounds to determine an appropriately sized bone plug to place in the scaphoid hole. Using an appropriately size hollow mill, such as hollow drill 430, 440, and 450 of FIG. 9, a plug of bone is removed from the radius. In a preferred embodiment, hollow drills 430, 440 and 450 are 3.0 mm, 3.4 mm and 3.8 mm drills, respectively. One of plug pin 400, 410 or 420 may be used to remove the bone plug from the hollow mill.

A further improvement of the fixation of the scapholunate reconstruction may be obtained by adding an additional step to the foregoing procedure, in an alternative method of the present invention. In this embodiment, an interference screw may or may not be used to secure a portion of graft 40 adjacent a sidewall of scaphoid tunnel 12. As shown in FIG. 23, distal arms 41 and 42 of graft 40, extending out of scaphoid tunnel 12 at its aperture at radial surface 11 of scaphoid 10, are taken and looped around the peripheral surface of scaphoid 10, up across the dorsal surface of scaphoid 10, across scapholunate joint 30, to the dorsal surface of lunate 20. Distal arms 41 and 42 of graft 40 are again pulled taught to close scapholunate gap 30, and are then secured to the dorsal surface of scaphoid 10 and/or the dorsal surface of lunate 20, using suture through drill holes, or a conventional suture anchor.

This alternative method of the present invention adds a second layer of reconstruction of the dorsal portion of the scapholunate ligament, and results in a distal sling portion of graft 40 that rigidly holds the base of scaphoid 10 to lunate 20, with one arm of the sling portion fixed in a more dorsal position and one arm in a more palmar position. This adds additional constraint to scapholunate joint 30, and adds additional stability to restrict axial rotation (i.e., pronation/supination movement) of scaphoid 10. With the two limbs of ligament reconstruction thus created, one through scapholunate joint 30 and one dorsally, there are two limbs of fixation present, separated by a distance, yielding improved resistance to torque and abnormal rotation of scaphoid 10 about its long axis. Moreover, this alternative method of the present invention restores significant stability to scapholunate joint 30, allows nearly normal flexion/extension of scaphoid 10 to occur in relation to lunate 20, while constraining any large dorsal translation of the base of scaphoid 10 and inhibiting gapping between scaphoid 10 and lunate 20. In addition, the fixation is secure enough to allow early mobilization of scapholunate joint 30 with rigid fixation of a sizeable graft 40 between two very small carpal bones.

An alternative method and associated apparatus for securing a suture tied to distal arms 41 and 42 of graft 40 adjacent dorsal surface of lunate 20, which may be used with or without a supplemental suture anchor, will now be described. In particular, a generally button-shaped suture anchor of the present invention is provided for use in cooperation with cannulated lunate screw 110.

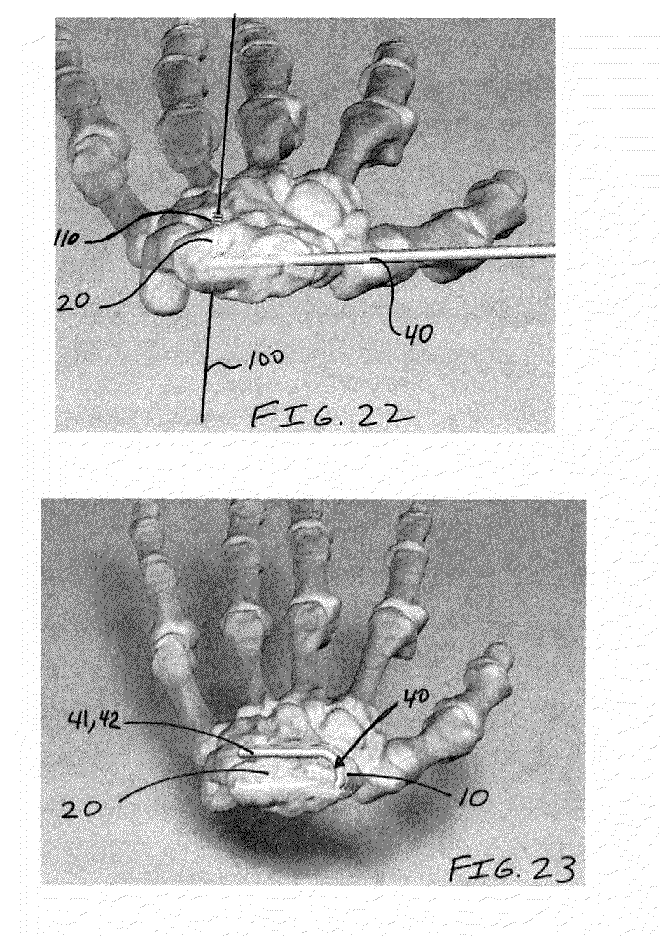

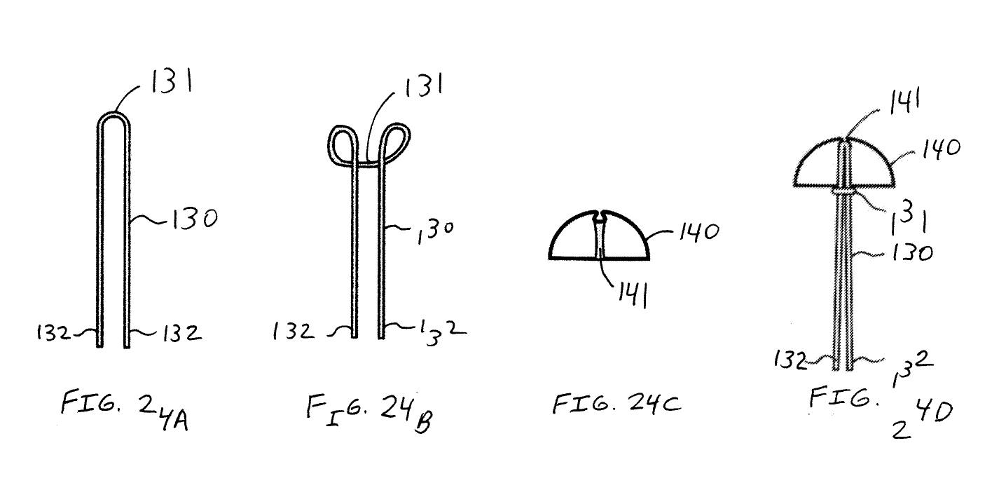

After threading cannulated lunate screw 110 into transverse secondary lunate hole 23, a pin is passed palmary to dorsally through the central hole of the lunate screw. This pin has a small loop of suture attached to a dorsal end thereof. Alternatively, the tendon wire used in the previous step to guide the lunate screw can be produced with a small loop of suture on the trailing portion to accomplish the same purpose. Referring to FIGS. 24A through 24D, a double armed suture 130 (i.e., a suture with a needle on each distal end 132) is passed through the loop of suture or wire attached to the pin. The pin is then withdrawn out palmar side 24 of lunate 20, pulling central loop 131 of double armed suture 130 through the central hole of cannulated lunate screw 110. On palmar side 24 of lunate 20, central loop 131 is retrieved. The loop attached to the end of the pin is then cut, freeing the loop of double armed suture 131. A second piece of heavy suture which is too large to pass through the central hole of the lunate screw 110 is then tied around the loop to anchor it on the palmar side when the arms of the suture 130 are sutured into the tendon graft dorsally, tightened and tied together. Alternatively, the suture can be folded back on itself as shown in FIG. 24B and then locked around a suture button 140. as shown in FIG. 24D.

As shown in FIG. 24C, a substantially hemispherical, button-shaped suture anchor 140 is provided. A central hemispherical groove 141 is provided to maintain an associated suture proximate an apex of suture anchor 140, and to keep the associated suture from sliding off in either direction from central groove 141. In a preferred embodiment, button-shaped suture anchor 140 is constructed of a biocompatible material such as metal, metal alloy, bioabsorbable material, PEEK, or other plastic material. Alternatively, this button could be spherical, flat, disc shaped, or other shapes.

Next, as shown in FIG. 24D, double armed suture 130 is fixed about button-shaped suture anchor 140, buy passing both loops of suture 130 about central groove 141 and then through central loop 131, with suture anchor 140 locked in place as distal ends 132 of suture 130 are tightened, thus forming a Lark's Head knot with suture 130 about suture anchor 140.

Distal ends 132 of suture 130 are then pulled taut through the dorsal side of lunate screw 110, anchoring suture anchor 140 and, in turn, central loop 131 against palmar surface 24 of lunate 20 (or the palmar end of lunate screw 110, if it is extending out beyond palmar surface 24 of lunate 20). The distal needles of suture 130 are then sewed into graft 40, tightened and tied to lock the distal arms 41 and 42 of graft 40 down against dorsal surface 21 of lunate 20.

Yet another alternative method and associated apparatus for securing a suture tied to distal arms 41 and 42 of graft 40 to the dorsal surface of lunate 20, which may be used with or without a supplemental suture anchor, will now be described. In particular, a generally bead-shaped suture anchor of the present invention is provided for use in conjunction with cannulated lunate screw 110.

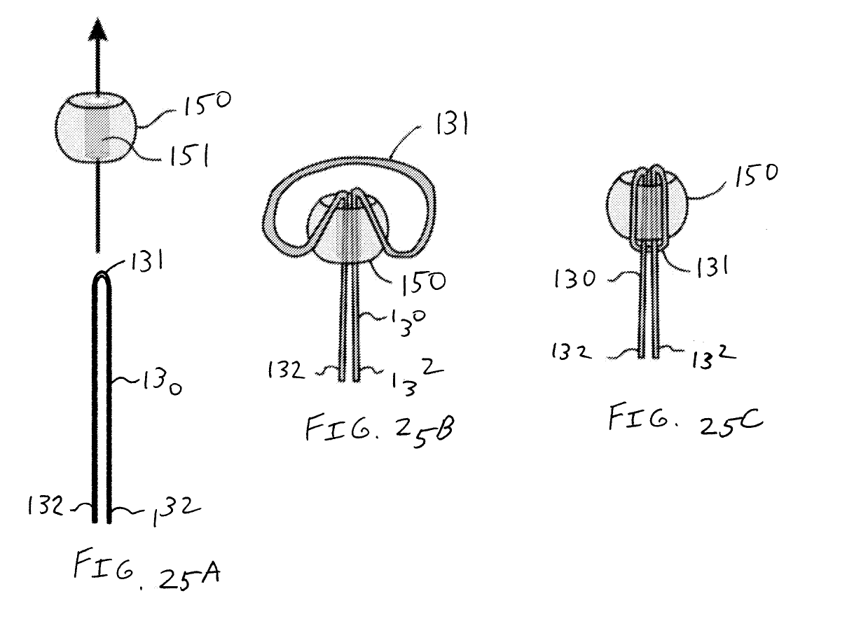

Referring to FIGS. 25A, central loop 131 of double armed suture 130 is passed first dorsal to palmar through the central channel of the lunate screw to exit palmarly, and then threaded through vertical channel 151 extending through bead-shaped suture button 150. Next, as shown in FIG. 25B, central loop 131 is widened to enable it to be pulled down around the exterior of bead-shaped suture button 150. Next, as shown in FIG. 25C, loop 131 is passed around the back of the button 150 and free ends 132 are pulled taut, forming a Lark's Head knot and securing suture 130 to bead-shaped suture button 150. In a preferred embodiment, bead-shaped suture anchor 150 is constructed of a biocompatible material such as metal, metal alloy, bioabsorbable material, PEEK, or other plastic material.

Distal ends 132 of double-armed suture 130 are then tightened and pulled taut from the dorsal side, anchoring suture anchor 150 and, in turn, central loop 131 against palmar surface 24 of lunate 20 (or the palmar end of lunate screw 110, if it is extending out beyond the palmar surface 24 of lunate 20). The distal needles of suture 130 are then sewed into graft 40, tightened and tied to lock the distal arms 41 and 42 of graft 40 down against the surface of lunate 20.

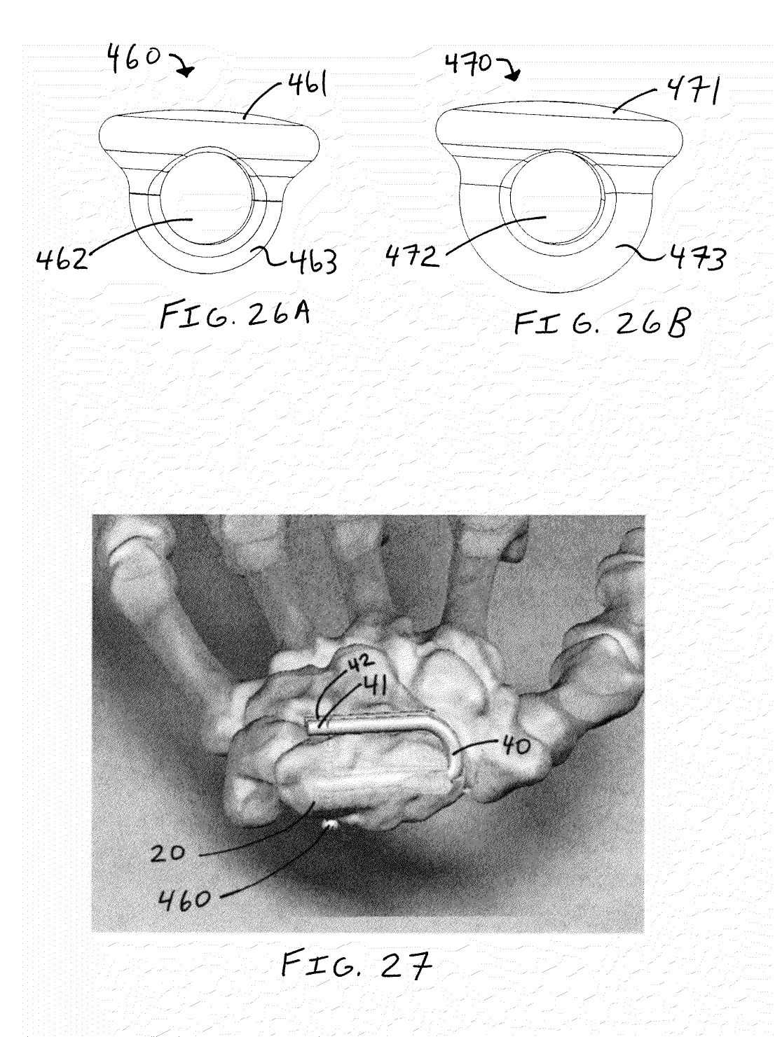

Additional alternative suture anchors 460 and 470 are shown in FIGS. 26A and 26B. Though slightly different in size and shape, suture anchors 460 and 470 share the same basic construction, having disc-shaped heads 461 and 461, arcuate bottom loops 463 and 473, and apertures 462 and 472, respectively. Suture anchor 460 is shown in FIG. 27, positioned with bottom loop 463 drawn into to the palmar side of vertical lunate hole 23 by a suture extending through the hole and sewn into distal arms 41 and 42 of graft 40 to secure graft 40 in place at the dorsal side of vertical lunate hole 23.

As an alternative to a suture button, a heavy strand of Ethibond® or other suitable suture may be tied securely to the loop of suture in the palmar incision to create a knot that is sized to be unable to pass through vertical lunate hole 23 or the cannulated channel 115 in lunate screw 110. Such polyester sutures are considered to be of sufficient strength to go up the lunate hole.

The present invention also includes kits of components, comprising combinations of several of the previously described implements. For example, any permutation or combination of two or more of any of the above-identified elements may be combined in kit form. Specific or assorted sizes of cannulated and non-cannulated drills, hexagonal or other form of drivers, and associated standard or quick-change handles may likewise further be included in any of these kit combinations.

By way of example, scapholunate reconstruction system kit 300 is shown in FIG. 9 as comprising tray 301, having a plurality of indentations for retaining implements and implants employed to perform the scapholunate reconstruction of the present invention. In particular, tray 301 holds, amongst other items, E-shaped guide 50; drill guide sleeves 80, 81; 2.0 mm drill 90; K-wires/guide pins 100; lunate screws 110; interference screws 120; suture beads 150; graft pusher 180; tendon stripper 310; tendon wire 320; guide pin drill guide 330; drill guide locator sleeve 350; cannulated drill 360; handle 370; pin depth gauge 380; tap 390; plug pins 400, 410 and 420; hollow drills 430, 440 and 450; and hex driver 480.

The preceding description and drawings merely explain the invention and the invention is not limited thereto, as those of ordinary skill in the art who have the present disclosure before them will be able to make changes and variations thereto without departing from the scope of the present invention.

1. A method for addressing instability of a scapholunate joint of a wrist, the scapholunate joint being an articulating region between a scaphoid and a lunate of the wrist, the method comprising the steps of:
obtaining a graft;
positioning at least a portion of the graft intramedullary to the scaphoid;
positioning at least a portion of the graft through the scapholunate joint; and
positioning at least a portion of the graft intramedullary to the lunate.

2. (canceled)

3. (canceled)

4. The method according to claim 1, further comprising the step of securing at least a portion of the graft intramedullary to the lunate.

5. The method according to claim 1, further comprising the step of securing at least a portion of an extramedullary portion of the graft to an extramedullary portion of the scaphoid.

6. The method according to claim 1, further comprising the step of securing at least a portion of an extramedullary portion of the graft to an extramedullary portion of the lunate.

7. (canceled)

8. (canceled)

9. (canceled)

10. (canceled)

11. (canceled)

12. (canceled)

13. (canceled)

14. The method according to claim 1, further comprising the step of advancing a guide pin into the scaphoid and substantially along a rotational center of the scaphoid.

15. The method according to claim 14, wherein the step of advancing the guide pin further comprises the steps of:
obtaining a guide having an associated referencing arm configured to locate an entry point for the guide pin a predetermined distance from a surface of the scaphoid;
positioning the referencing arm adjacent a surface of the scaphoid; and
advancing the guide pin through an aperture of the guide.

16. The method according to claim 14, further comprising the step of extending a cannulated drill along the guide pin to drill a hole through the scaphoid and at least partially into the lunate.

17. (canceled)

18. The method according to claim 61, further including the step of extending at least a portion of a drill through the first drill sleeve associated with the at least one outer arm, into the lunate, through the hooked end of the inner arm, and out an opposing side of the lunate.

19. (canceled)

20. The method according to claim 59, further comprising the step of extending a flexible line through the second lunate bone tunnel.

21. (canceled)

22. The method according to claim 69, further comprising the step of looping a portion of the graft through the looped portion of the flexible line, configuring the graft into a looped end, a first distal arm and a second distal arm.

23. (canceled)

24. (canceled)

25. (canceled)

26. The method according to claim 51, further comprising the step of placing a retaining member within at least a portion of the lunate for fixation of at least a portion of the graft within the first lunate bone tunnel.

27. The method according to claim 76, wherein the retaining member comprises a cannulated screw.

28. The method according to claim 1, further comprising the step of securing at least a portion of the graft intramedullary to the scaphoid.

29. The method according to claim 28, wherein an interference screw is used to secure at least a portion of the graft intramedullary to the scaphoid.

30. The method according to claim 28, wherein a bone plug is used to secure at least a portion of the graft intramedullary to the scaphoid.

31. The method according to claim 6, wherein at least a portion of the graft is secured extramedullary to the scaphoid.

32. The method according to claim 31, wherein at least one of a suture and a suture anchor is used to secure at least a portion of the graft to the scaphoid.

33. (canceled)

34. The method according to claim 32, wherein at least a portion of an extraosseous portion of the graft is secured proximate a retaining member securing at least a portion of the graft intramedullary to the lunate.

35. The method according to claim 34, wherein the graft is secured to a suture extending through a cannula of the retaining member.

36. The method according to claim 35, wherein the suture is retained on an opposing side of the retaining member by a suture anchor.

37. (canceled)

38. A method for addressing instability of a scapholunate joint of a wrist, the scapholunate joint being an articulating region between a scaphoid and a lunate of the wrist, the method comprising the steps of:
obtaining a graft;
creating a first hole through at least a portion of the scaphoid;
creating a second hole into at least a portion of the lunate, the first hole and the second hole being substantially alignable across the scapholunate joint;
creating a third hole into at least a portion of the lunate, the third hole communicating with the second hole;
positioning a portion of the graft within the first hole and the second hole, with an end of the graft proximate an intersection of the second hole and the third hole;
positioning at least a portion of a retaining member within the third hole to secure the end of the graft intramedullary to the lunate; and
securing at least a portion of the graft to at least one of: a) intramedullary to the scaphoid; and b) extramedullary to at least one of the lunate and the scaphoid.

39. A drill guide facilitating performing a portion of a reconstructive procedure for addressing instability of a scapholunate joint of a wrist, the scapholunate joint being an articulating region between a scaphoid and a lunate of the wrist, the drill guide comprising an inner arm, a first outer arm operably coupled to the inner arm, and a first drill sleeve having a first longitudinal axis extending through the first outer arm, at least a portion of the inner arm being sized for insertion into a first hole through the lunate and a second hole through at least a portion of the scaphoid, such that an extension of the first longitudinal axis substantially intersects a distal portion of the inner arm, the inner arm and first outer arm being in a spaced relationship so as to permit the first drill sleeve and a drill bit to be positioned substantially adjacent an outer surface of the lunate upon insertion of the inner sleeve through the first hole, across the scapholunate joint and into the second hole.

40. The drill guide according to claim 39, further comprising a second outer arm operably coupled to the central arm, the first outer arm and second outer arm being disposed on an opposing sides of the central arm.

41. The drill guide according to claim 40, wherein the first drill sleeve is insertable through an aperture of at least one of the first outer arm and the second outer arm.

42. (canceled)

43. (canceled)

44. The drill guide according to claim 39, wherein the central arm is constructed of a substantially radio-opaque material.

45. The drill guide according to claim 40, wherein at least a portion of at least one of the first and second outer arms is constructed of a substantially radiolucent material.

46. A surgical guide wire comprising:
a first substantially rigid member having a trailing end;
a substantially flexible member having a leading end and a trailing end, wherein the leading end of the substantially flexible member is affixed to the first substantially rigid member proximate the leading end of the substantially flexible member and over at least a portion of the trailing end of the substantially rigid member; and
a second substantially rigid member having a leading end and a trailing end, wherein the leading end of the second substantially rigid member is affixed to the trailing end of the substantially flexible member.

47. (canceled)

48. The surgical guide wire according to claim 47, wherein the first substantially rigid member and the second substantially rigid member are of different diameters in size.

49. The surgical guide wire according to claim 47, further comprising at least one of a substantially flexible wire and a suture, formed into a loop and affixed to the trailing end of the second substantially rigid member.

50. A kit of implements for performing a reconstructive procedure for addressing instability of a scapholunate joint of a wrist, the scapholunate joint being an articulating region between a scaphoid and a lunate of the wrist, the kit comprising at least the following:
a first guide configured to facilitate the drilling of a first hole through the scaphoid and a second, substantially collinear hole into the lunate, the first guide having an associated referencing arm configured to locate an entry point for a guide pin a predetermined distance from a surface of the scaphoid;
a second guide configured to facilitate the drilling of a third hole into at least a portion of the lunate, at least a portion of the third hole communicating with at least a portion of the second hole, the second guide comprising an inner arm, a first outer arm operably coupled to the inner arm, and a first drill sleeve having a first longitudinal axis extending through the first outer arm at least a portion of the inner arm being sized for insertion into the first hole and the second hole, such that an extension of the first longitudinal axis substantially intersects a distal portion of the inner arm, the inner arm and first outer arm being in a spaced relationship so as to permit at least one of a drill sleeve and a drill bit to be positioned substantially adjacent an outer surface of the lunate upon insertion of the inner sleeve through the first hole, across the scapholunate joint and into the second hole;
a retaining member configured to be placed into the third hole and capable of securing an end of a tendon graft intramedullary to the lunate
a graft pushing implement;
at least one cannulated drill bit; and
a tendon wire positionable through the third hole and through a looped end of the tendon graft.

51. The method according to claim 1, further comprising the steps of:
creating a scaphoid bone tunnel extending through at least a portion of the scaphoid and having a aperture within an articulating surface of the scapholunate joint; and
creating a first lunate bone tunnel extending through the articulating surface of the scapholunate joint and into at least a portion of the lunate.

52. The method according to claim 51, wherein the first lunate bone tunnel has a closed terminal end within an intramedullary portion of the lunate.

53. The method according to claim 51, wherein the scaphoid bone tunnel and the first lunate bone tunnel are substantially collinearly alignable.

54. The method according to claim 51, wherein the scaphoid bone tunnel is directed proximate to a central axis of rotation of the scaphoid relative to the lunate.

55. The method according to claim 51, wherein the scaphoid bone tunnel and the first lunate bone tunnel are positioned to align dorsal and palmar surfaces of the scaphoid and lunate when the scaphoid bone tunnel and the first lunate bone tunnel are substantially collinearly aligned.

56. The method according to claim 51, further comprising the step of creating a second lunate bone tunnel extending through at least a portion of the lunate, the second lunate bone tunnel intersecting the first lunate bone tunnel within the lunate and extending proximate an extraarticular surface of the lunate.

57. The method according to claim 56, wherein the second lunate bone tunnel is substantially perpendicular to the first lunate bone tunnel.

58. The method according to claim 56, wherein the second lunate bone tunnel intersects the first lunate bone tunnel proximate an endpoint of the first lunate bone tunnel within the lunate.

59. The method according to claim 56, wherein the second lunate bone tunnel extends from dorsal to palmar surfaces of the lunate.

60. The method according to claim 51, further comprising the step of obtaining a drill guide, the drill guide comprising:
an inner arm, at least a portion of the inner arm being sized for insertion into the scaphoid bone tunnel, across the scapholunate joint, and into the first lunate bone tunnel;
at least one outer arm operably connected to the inner arm and extendable along an outer surface of the lunate;
at least one drill guide allowing passage of a drill or pin therethrough to create a second lunate bone tunnel extending through at least a portion of the lunate;
the at least one drill guide being positioned along the at least one outer arm so as to cause the second lunate bone tunnel to intersect the first lunate bone tunnel.

61. The method according to claim 60, wherein said inner arm has a hooked end having an eyelet, wherein the second lunate bone tunnel intersects the eyelet.

62. The method according to claim 60, wherein the second lunate bone tunnel is proximate a terminal end of the first lunate bone hole.

63. The method according to claim 61, wherein the eyelet is configured to allow placement of the drill or pin therethrough.

64. The method according to claim 61, wherein the eyelet includes an open slot permitting a wire or pin extending through the eyelet to be removed from the eyelet.

65. The method according to claim 60, wherein the at least one outer arm comprises a first outer arm extendable along a dorsal surface of the lunate and a second outer arm extendable along a palmar surface of the lunate.

66. The method according to claim 60, wherein the drill guide is configured to permit the allows second bone tunnel to extend from a dorsal surface of the lunate to a palmar surface of the lunate.

67. The method according to claim 65, wherein at least one of the first arm and the second arm includes an aperture configured to capture a drill bit extending therethrough, thereby avoiding injury to adjacent soft tissue.

68. The method according to claim 60, wherein at least a portion of the at least one outer arm is constructed of a substantially radiolucent material.

69. The method according to claim 20, further comprising the step of withdrawing a looped portion of the flexible line out of the first lunate bone tunnel, across the scapholunate joint and out of the scaphoid bone tunnel.

70. The method according to claim 69, wherein a hook is employed to withdraw the looped portion of the flexible line.

71. The method according to claim 22, further comprising the step of positioning the graft within the scaphoid bone tunnel, across the articulating surface of the scapholunate joint, and within the first lunate bone tunnel.

72. The method according to claim 71, the step of positioning the graft comprises positioning the looped portion of the graft proximate to an intersection of the first lunate bone tunnel and a second lunate bone tunnel.

73. The method according to claim 71, wherein the step of positioning the graft comprises pulling opposing ends of the flexible line to, in turn, position the looped portion of the graft proximate to an intersection of the first lunate bone tunnel and a second lunate bone tunnel.

74. The method according to claim 22, further comprising the step of pushing the looped end of the graft through the scaphoid bone tunnel, across the scapholunate joint and into the first lunate bone tunnel.

75. The method according to claim 56, further comprising the steps of:
obtaining a graft;
forming the graft into a loop with two distal arms;
positioning the graft within the lunate bone tunnel, across the scapholunate joint, and within the first scaphoid bone tunnel; and
positioning the loop of the graft proximate an intersection of the first lunate bone tunnel and the second lunate bone tunnel.

76. The method according to claim 75, further comprising the steps of:
passing a retaining member through the second lunate bone tunnel; and
passing the retaining member through the loop of graft material to, in turn, secure the loop within the first lunate bone tunnel.

77. The method according to claim 76, wherein the step of passing a retaining member through the second lunate bone tunnel comprises the steps of:
placing a guide member through the loop of graft; and
passing the retaining member the said guide member.

78. The method according to claim 77, wherein at least a portion of the guide member comprises a flexible line.

79. The method according to claim 77, wherein at least a portion of the guide member comprises a pin.

80. The method according to claim 38, wherein the graft is formed into a loop with a looped end and a first and second distal arm, and wherein the step of positioning a portion of the graft comprises positioning the graft such that the looped end is proximate an intersection of the second hole and the third hole.

81. The method according to claim 80, wherein the step of positioning at least a portion of the retaining member within the third hole comprises passing the retaining member through the looped end of the graft.
